(19) United States
(12) Patent Application Publication
(10) Pub. No.: US 2004/0028749 A1
(43) Pub. Date: Feb. 12, 2004

(54) Treating inflammation and associated complications

(76) Inventors: Arne Slungaard, Saint Paul, MN (US); Stanley L. Hazen, Pepper Pike, OH (US); Husam M. Abu-Soud, Rochster Hills, MI (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/427,271

(22) Filed: May 1, 2003

Related U.S. Application Data
(63) Continuation-in-part of application No. PCT/US01/46934, filed on Nov. 2, 2001.
(60) Provisional application No. 60/245,812, filed on Nov. 3, 2000.

(51) Int. Cl. A61K 33/00

(52) U.S. Cl. 424/600

(57) ABSTRACT

The invention provides methods and materials related to the treatment of inflammation. Such inflammations include, without limitation, those inflammations mediated by eosinophils and/or neutrophils as well as those inflammations associated with peroxidase activity. The invention also provides methods and materials related to the treatment of complications associated with eosinophil-mediated inflammations and/or neutrophil-mediated inflammations. Specifically, the invention involves increasing the amount of pseudohalides in a mammal exhibiting inflammation and/or an associated condition.

### CROSS REFERENCE TO RELATED APPLICATIONS

This application is a continuation-in-part of, and claims the benefit of priority to PCT Application PCT/US01/46934 filed Nov. 2, 2001, which claims the benefit of priority to U.S. Application No. 60/245,812, filed Nov. 3, 2000.

### BACKGROUND

1. Technical Field

The invention relates to methods and materials involved in treating inflammations such as those inflammations mediated by eosinophils and/or neutrophils as well as those inflammations associated with peroxidase activity.

2. Background Information

Diseases characterized by eosinophil mediated inflammation include nearly every type of acute and chronic allergic response, polyposis, rhinitis, and asthma. Asthma afflicts 5-8 percent of the United States population. Asthma is a highly prevalent hypersensitivity disease that causes considerable morbidity and mortality. It is characterized by bronchial infiltration with activated eosinophils (EOs) and consequent tissue damage and pathology. A critical component of the EO cytotoxic armamentarium is its highly active EO peroxidase (EPO), which is by weight the most abundant component of the EO specific granule. EPO is functionally and structurally distinct from the neutrophil myeloperoxidase (MPO), which uses H₂O₂ to oxidize chloride (Cl⁻) to hypochlorous acid (HOCl), a highly reactive but indiscriminately destructive bleaching oxidant. One known physiologic substrate of EPO, bromide (Br⁻) is oxidized by EPO to yield a similarly indiscriminately reactive and destructive hypohalous oxidant, hypobromous acid (HOBr).

### SUMMARY

The invention provides methods and materials related to the treatment of inflammation. Such inflammations include, without limitation, those inflammations mediated by EOs and/or neutrophils as well as those inflammations associated with peroxidase activity. For example, the invention provides methods and materials related to the treatment of EO-mediated inflammations and neutrophil-mediated inflammations. The invention also provides methods and materials related to the treatment of complications associated with EO-mediated inflammations and/or neutrophil-mediated inflammations.

In one aspect, the invention features a method for treating an EO-mediated inflammation in a mammal, the method includes administering a pseudohalide to the mammal under conditions such that the EO-mediated inflammation is reduced. The method also can include monitoring the level of the pseudohalide. The EO-mediated inflammation can be asthma, rhinitis, eczema, contact dermatitis, hypereosinophilic syndrome, or polyposis. The pseudohalide can have an oxidative reactivity with a peroxidase greater than that of Br⁻ (i.e., the pseudohalide is preferentially oxidized by a peroxidase over Br⁻). The pseudohalide, when oxidized by EPO, can form a product having lower toxicity than HOBr. The pseudohalide can be administered orally, nasally, or topically. The pseudohalide can be administered by injection or inhalation. The administration can include administering a pill or a dietary supplement containing the pseudohalide. The pseudohalide can be administered at a dose between 1 μg to 10 g/day.

In another embodiment, the invention features a method of reducing peroxidase catalyzed oxidation of a halide in a mammal (e.g., human), the method includes administering a pseudohalide to the mammal such that the pseudohalide contacts the peroxidase and reduces the oxidation of the halide. The method also can include monitoring the level of the pseudohalide. The peroxidase can be EPO, MPO, or LPO. The halide can be bromide, iodide, and chloride. The pseudohalide can be thiocyanate (SCN⁻), salts of SCN⁻, isothiocyanate, or salts of isothiocyanate.

Another embodiment of the invention features a method for treating a mammal (e.g., human) having bronchial constriction, the method includes administering a pseudohalide to the mammal such that the bronchial constriction is reduced. The method also can include monitoring the level of the pseudohalide. The pseudohalide can be SCN⁻, salts of SCN⁻, isothiocyanate, or salts of isothiocyanate. The pseudohalide can have an oxidative reactivity with a peroxidase greater than that of Br⁻. The pseudohalide, when oxidized by EPO, can form a product having lower toxicity than HOBr. The pseudohalide can be administered orally, nasally, or topically. The pseudohalide can be administered by injection or inhalation. The administration can include administering a pill or a dietary supplement containing the pseudohalide.

Another embodiment of the invention features a method for increasing the amount of NO in the tissue of a mammal, the method including administering a pseudohalide to the mammal such that the amount is increased. The method also can include monitoring the level of the pseudohalide. The pseudohalide can be administered orally, nasally, topically, or by injection or inhalation. The pseudohalide can be a pill. The pseudohalide can be a dietary supplement.

Another aspect of the invention features a bronchial inhaler device containing an aerosolizable form of a pseudohalide. The pseudohalide can be SCN⁻, salts of SCN⁻, isothiocyanate, or salts of isothiocyanate.

In another embodiment, the invention features a method of identifying a mammal that can benefit from treatment with a pseudohalide. The method involves determining the level of the pseudohalide in a biological sample from the mammal and then identifying the mammal as one that can benefit from treatment with the pseudohalide if said level of the pseudohalide is reduced relative to that of a control sample. The pseudohalide can be thiocyanate, salts of thiocyanate, isothiocyanate, and salts of isothiocyanate. The biological sample can be bronchoalveolar lavage, serum, plasma, urine, tissue biopsy, sputum, induced sputum, blood, pericardial fluid, pleural fluid, and cerebrospinal fluid.

In another embodiment, the invention features a process for aiding in the diagnosis of a disease condition. The process involves manufacturing a diagnostic device comprising a thiocyanate-reactive reagent that is capable of reacting with thiocyanate in a biological sample to form a detectable product, and then selling the diagnostic device to an organization involved in managing or providing healthcare.

In another embodiment, the invention features a process for aiding in the treatment of a disease condition. The process involves manufacturing a pharmaceutical composition comprising a pseudohalide having an oxidative reactivity with a peroxidase greater than that of bromide and a lower degree of toxicity than HOBr, and then selling the pharmaceutical composition to an organization involved in managing or providing healthcare. The pseudohalide can be thiocyanate, salts of thiocyanate, isothiocyanate, and salts of isothiocyanate.

In another embodiment, the invention provides for the use of a pseudohalide in the manufacture of a medicament for the treatment of eosinophil-mediated inflammation in mammal such that administration of the pseudohalide is effective for reducing eosinophil-mediated inflammation. The pseudohalide can be thiocyanate, salts of thiocyanate, isothiocyanate, and salts of isothiocyanate.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a graph illustrating the SCN⁻-mediated inhibition of HOBr production by EPO.

FIG. 2A is a ¹³C nuclear magnetic resonance spectrum of the reaction products of the EPO/SCN⁻/H₂O₂ system at pH 7.4 without EPO.

FIG. 2B is a ¹³C nuclear magnetic resonance spectrum of the reaction products of the EPO/SCN⁻/H₂O₂ system at pH 7.4.

FIG. 2C is a ¹³C nuclear magnetic resonance spectrum of the reaction products of the EPO/SCN⁻/H₂O₂ system at pH 7.4 after 16-hour incubation at 4° C.

FIG. 2D is a ¹³C nuclear magnetic resonance spectrum of the reaction products of the EPO/SCN⁻/H₂O₂ system at pH 6.0.

FIG. 2E is a ¹³C nuclear magnetic resonance spectrum of the reaction products of the EPO/SCN⁻/H₂O₂ system at pH 6.0 after 16 hour incubation at 4° C.

FIG. 3A is a negative ion electrospray ionization mass spectrum of the reaction products of the EPO/SCN⁻/H₂O₂ system in the absence of EPO at m/z range of 70-80.

FIG. 3B is a negative ion electrospray ionization mass spectrum of the reaction products of the EPO/[¹²C]SCN⁻/H₂O₂ system at m/z range of 70-80.

FIG. 3C is a negative ion electrospray ionization mass spectrum of the reaction products of the EPO/[¹³C]SCN⁻/H₂O₂ system at m/z range of 70-80.

FIG. 3D is a negative ion electrospray ionization mass spectrum of the reaction products of the EPO/SCN⁻/H₂O₂ system in the absence of EPO at m/z range of 40-50.

FIG. 3E is a negative ion electrospray ionization mass spectrum of the reaction products of the EPO/[¹²C]SCN⁻/H₂O₂ system at m/z range of 40-50.

FIG. 3F is a negative ion electrospray ionization mass spectrum of the reaction products of the EPO/[¹³C]SCN⁻/H₂O₂ system at m/z range of 40-50.

FIG. 4A is a collision-induced dissociation tandem mass spectrum of the m/z 74 parent ion product of the EPO/SCN⁻/H₂O₂ system.

FIG. 4B is a collision-induced dissociation tandem mass spectrum of the m/z 42 parent ion product of the EPO/SCN⁻/H₂O₂ system.

FIG. 5 is a graph illustrating the inactivation of RBC enzymes by products of the EPO/SCN⁻/H₂O₂ system.

FIG. 6 is a graph illustrating the effect of increasing SCN⁻ concentration on Br⁻-based toxicity in an endothelial cell model.

FIG. 7 is is a bar graph illustrating the effect of SCN⁻ on generation of HETEs from PAPC phospholipid vesicles by the EPO/H₂O₂NO₂⁻ system.

FIG. 8A is a graph illustrating NO decay by autoxidation.

FIG. 8B is a graph illustrating accelerated NO decay in the presence of an O₂^{.-} generating system.

FIG. 8C is a graph illustrating the consumption of NO following addition of either EPO or LPO. FIG. 8D is a graph illustrating SCN⁻-mediated attenuation of peroxidase-mediated NO consumption.

FIG. 8E is a graph illustrating SCN⁻-mediated attenuation of peroxidase-mediated NO consumption.

FIG. 9 is a graph illustrating the effect of SCN⁻ on tracheal ring relaxation. FIG. 10 is a graph illustrating the effects of 10 and 100 μM SCN⁻ on peroxidase-dependent inhibition of NO-dependent bronchodilation.

FIG. 11 is a graph comparing the serum SCN⁻ concentrations in normals and eosinophilics.

FIG. 12 is a graph comparing the serum SCN⁻ concentrations in normals and asthmatics.

FIG. 13A is a comparison of the rate of NO consumption in a 6 μM NO/0.2 M sodium phosphate buffer (pH 7.0) stirred solution at 25° C. under air by (i) autoxidation (dotted line) and (ii) following addition of H₂0₂ and MPO (solid line) determined by an NO-selective electrode.

FIG. 13B is a recording by an NO-selective electrode illustrating the consumption of NO in a 6 μM NO/0.2 M sodium phosphate buffer (pH 7.0) stirred solution at 25° C. under air following addition of MPO then H₂0_{2.}

FIG. 13C is a recording by an NO-selective electrode illustrating the consumption of NO in a 6 μM NO/0.2 M sodium phosphate buffer (pH 7.0) stirred solution at 25° C. under air following H₂0₂ and then EPO addition.

FIG. 13D is a recording by an NO-selective electrode illustrating the consumption of NO in a 6 μM NO/0.2 M sodium phosphate buffer (pH 7.0) stirred solution at 25° C. under air following H₂0₂ and then LPO addition.

FIG. 14 is a recording by an NO-selective electrode demonstrating the effect of MPO on NO consumption in the presence of a superoxide generating system.

FIG. 15 are two tracings from four typical tracheal rings (R1-R4) harvested from rats, transduced under tension, and contracted with bethanecol to 50-75% of tension illustrating inhibition of NO-dependent bronchodilation by peroxidase-H₂O₂ systems.

FIG. 16 is a tracing illustrating time-dependent NO-mediated relaxation of preconstricted tracheal rings and the requirement for the combined presence of each component of the peroxidase-H₂O₂ system for attenuation of NO-mediated tracheal relaxation.

FIG. 17 is a set of NO concentration-response curves for intact tracheal rings showing that peroxidase-H₂O₂ systems prevent NO-mediated relaxation. Data represent the mean±SD of four independent experiments.

FIG. 18 is a graph demonstrating that EPO and LPO concentrations modulate NO-induced relaxation of intact tracheal rings. Data are the mean±SD of four experiments.

FIG. 19 is a stable isotope dilution GC-MS analysis of NO₂—Y in proteins recovered in endotracheal/bronchial aspirates from severe asthmatic and nonasthmatic subjects. Data points represent the mean of duplicate determinations of samples from each individual. Numbers in parentheses represent the number of subjects in each group. Mean values±SD for each group are shown.

FIG. 20A is a stable isotope GC-MS analysis of Br⁻ Y in proteins recovered in endotracheal/bronchial aspirates from severe asthmatic and nonasthmatic subjects. Data points represent the mean of duplicate determinations of samples from each individual. Numbers in parentheses represent the number of subjects in each group. Mean values±SD for each group are shown.

FIG. 20B is a stable isotope GC-MS analysis of Cl—Y in proteins recovered in endotracheal/bronchial aspirates from severe asthmatic and nonasthmatic subjects. Data points represent the mean of duplicate determinations of samples from each individual. Numbers in parentheses represent the number of subjects in each group. Mean values±SD for each group are shown.

FIG. 21A is a graph illustrating tyrosine nitration by PMA-activated human EOs in the presence or absence of 50 μM NaNO₂ at 37° C. for the indicated time intervals. Data represent the mean±SD of triplicate determinations for a characteristic experiment performed at least three times.

FIG. 21B is a graph illustrating superoxide anion production by activated EOs measured following EO activation in the absence NaNO₂. Data represent the mean±SD of triplicate determinations for a characteristic experiment performed at least three times.

FIG. 22 is a series of bar graphs illustrating EO-dependent nitration and halogenation of phenolic targets. Data represent the mean±SD of triplicate determinations for a characteristic experiment performed at least three times.

FIG. 23A is a graph illustrating human EO modification of HPA at physiologically relevant levels of nitrite (NO₂⁻). Data represent the mean±SD of triplicate determinations for a characteristic experiment performed at least three times.

FIG. 23B is a graph illustrating neutrophil modification of HPA at physiologically relevant levels of NO₂⁻. Data represent the mean±SD of triplicate determinations for a characteristic experiment performed at least three times.

FIG. 24 is a graph illustrating the amounts of NO₂-HPA, Br-HPA, and Cl-HPA formed by phorbol ester-stimulated human EOs at the indicated rates of NO release. Data represent the mean±SD of triplicate determinations for a characteristic experiment performed at least three times.

FIG. 25 is a graph illustrating the effect of EPO inhibitor, H₂O₂ scavenger, and SOD on EO-mediated aromatic nitration reactions.

FIG. 26 is a graph illustrating the effect of SCN-supplementation on the levels of Br-Y (top panel) and Cl—Y (bottom panel) in guinea pigs.

### DETAILED DESCRIPTION

In general, methods of the invention include administering a psuedohalide to a mammal for treating inflammations including, without limitation, those inflammations mediated by EOs and/or neutrophils or inflammations associated with peroxidase activity, and associated complications (e.g., bronchial constriction). The term “EO-mediated inflammation” as used herein refers to any inflammation associated with the presence of EOs. Conditions involving EO-mediated inflammation can be identified using medically accepted diagnostic techniques. For example, methacholine challenge can be used to identify an asthmatic patient. Medical conditions involving EO-mediated inflammation include, without limitation, asthma, polyposis, rhinitis, eczema, contact dermatitis, hypereosinophilic syndrome, and other allergy type illnesses. Other examples of EO-mediated inflammations include, without limitation, those conditions described by Zucher-Franklin in Chapter 92 of Hematology (third edition, edited by Williams et al., McGraw-Hill Book Company (1983)) and page 1012 of Cecil's Textbook of Medicine (17th Edition, edited by James B. Wyngaarden and Lloyd H. Smith, Jr. (1985) W. B. Saunders Company, Philadelphia, Pa.

The term “neutrophil-mediated inflammation” as used herein refers to any inflammation associated with the presence of neutrophils. Medical conditions involving neutrophil-mediated inflammation include, without limitation, ischaemia/reperfusion, thrombosis, autoimmune diseases, and bacterial, fungal, and viral infections. Conditions involving neutrophil-mediated inflammation can be identified using medically accepted diagnostic techniques.

Use of pseudohalides, and in particular thiocyanate, to treat asthma and other allergic conditions is advantageous over other approaches because thiocyanate is naturally found in vegetative matter and is virtually innocuous, even at serum levels up to 10 times those normally found in mammals. Manipulation of potential substrates for peroxidase oxidation by dietary or other means also reduces non-specific effects that may occur by global inhibition of peroxidase activity by peroxidase inhibitors.

Pseudohalide

As indicated above, inflammations such as EO-mediated inflammations, neutrophil-mediated inflammations, and inflammations associated with peroxidase activity (e.g., EPO, myeloperoxidase (MPO), or lactoperoxidase (LPO) activity) as well as associated complications can be treated by administering a pseudohalide. The term “pseudohalide” as used herein refers to any nonhalide moiety capable of reacting as a substrate with peroxidase in the presence of the co-substrate H₂O₂ to form an oxidant product. Suitable pseudohalides are those that have an oxidative reactivity, i.e., a specificity constant (K_{cat}/Kₘ), with peroxidase greater than that of nitrite and bromide, and that form a product that is less toxic than HOBr or NO₂. when oxidized by a peroxidase (e.g., EPO, MPO, or LPO). Pseudohalides can be, without limitation, thiocyanate, isothiocyanate, and guaiacol. Pseudohalides of the invention also include, for example, salts of thiocyanate and isothiocyanate.

Pseudohalides such as thiocyanate are commercially available (e.g., from Sigma Chemical Co., St. Louis, Mo.). Thiocyanate derives from the rhodanese detoxification reaction between cyanide ion and thiosulfates. Other sources of thiocyanate, thiocyanate precursors such as isothiocyanate, and thiocyanate esters include plants in the Cruciferae family. Examples of Cruciferae plants include, without limitation, Brassica species such as cabbage, cauliflower, kale, brussel sprouts, broccoli, and kohlrabi. Thiocyanate also in present in cassaya, yams, millet, and sorghum. See Wook J L in Chemistry and Biochemistry of Thiocyanic Acid and its Derivatives (Newmann AA, ed), Academic Press, Orlando, Fla., page 156-221, 1975.

Pseudohalides such as thiocyanate can be formulated as a pharmaceutical composition suitable for administration to a mammal such as a human. Typically, a pseudohalide is mixed with a pharmaceutically acceptable carrier or excipient. Various pharmaceutically acceptable carriers can be used, including for example physiological saline or other known carriers appropriate to specific routes of administration. Preparations for administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water as well as alcohol, saline, and buffered solutions. Preservatives, flavorings, and other additives such as, for example, antimicrobials, anti-oxidants, chelating agents, inert gases, and the like may also be present.

Tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). The tablets can be coated by methods known in the art. Preparations for oral administration can be formulated to give controlled or gradual release of the compound. Biocompatible, biodegradable lactide polymer, lactide/glycolide copolymer, or polyoxethylene-polyoxypropylene copolymers are examples of excipients for controlling the release of the pseudohalides.

Liquid preparations for oral administration can take the form of, for example, solutions, syrups or suspension, or they can be presented as a dry product for constitution with saline or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Pharmaceutical compositions for inhalation can be in the form of a liquid solution, a gel, or a dry product. Inhalation formulations may be aqueous solutions containing, for example, polyoxyethylene-9-lauryl ether, glycocholate and deoxycholate, and may contain excipients such as lactose, if desired. Nebulised aqueous suspensions or solutions can include carriers or excipients to adjust pH and/or tonicity. Bronchial inhalers of the invention can include such formulations of pseudohalides. Nasal drops can be administered in the form of oily solutions.

Pharmaceutical compositions for parenteral administration include liquid solutions or suspensions in aqueous physiological buffer solutions, i.e., one that is similar in pH, is isotonic, or both similar in pH and isotonic, to a selected mammal, can be prepared as desired using standard methods. Formulations may contain common excipients as well as glycocholate for buccal administration. Suitable parenteral delivery systems include ethylene-vinyl acetate copolymer particles, osmotic pumps, implantable infusion systems, and liposomes. For topical administration, pseudohalide can be in the form of a cream or ointment.

Pharmaceutical preparations of pseudohalides are substantially free of contaminants. As used herein, the term “contaminant” refers to any compound or macromolecule that has a deleterious effect on a mammal upon repeated or a single exposure through ingestion, inhalation, injection or any means of contact. Contaminants can be pesticides, metals such as heavy metals, or toxins from bacteria, fungi, algae, plants, or animals. Pesticides are any substance or mixture of substances (chemical or biologic) that is intended to prevent, destroy, repell, or mitigate any pest. Heavy metals generally include those metals from periodic table groups IIA through VIA. Examples of heavy metals include aluminum, antimony (including stibine), arsenic (including arsine), barium, beryllium, bismuth, boron (including decaborane, diborane, pentaborane), cadmium, calcium, chromium, cobalt, copper, hafnium, iron, lead (including tetraethyl lead, tetramethyl lead), magnesium, manganese, mercury, molybdenum, nickel, osmium (tetroxide), platinum, rhodium, selenium (including hydrogen selenide), silver, tantalum, tellurium, thallium, tin, titanium (dioxide), uranium, vanadium, yttrium, zinc, and zirconium. Toxins from bacteria, fungi, algae, plants, and animals can be, without limitation, small or large molecular weight polypeptides, lipopolysaccharides, amines, lipids, steroids, aminopolysaccharides, quinines, or glycosides. Contaminants do not include the above discussed buffers, excipients, or carriers.

Treatment of Inflammation or Associated Complications

Methods for treating inflammations and associated complications (e.g., bronchial constriction) include, without limitation, administering a pseudohalide (e.g., thiocyanate (SCN⁻)) in an amount sufficient to (1) elevate thiocyanate concentration in serum, other bodily fluids, or the inflamed tissue, (2) reduce the amount of cytotoxic oxidants normally formed by peroxidases under conditions of low thiocyanate concentration, or (3) reduce nitric oxide (NO) consumption by peroxidases (e.g., EPO, MPO, and LPO). Cytotoxic oxidants include hypohalous acids derived from halides (e.g., HOBr and HOCl). For example, the amount of pseudohalide in lung tissue can be increased to treat an EO-mediated inflammation such as asthma. Without being bound to a particular mechanism, increasing serum blood levels of thiocyanate and/or locally manipulating thiocyanate concentrations (e.g., by aerosol inhalation of thiocyanate) may block both EPO-mediated oxidative protein modification/damage and scavenging of NO, a bronchodilator, and thereby diminish the signs and symptoms of asthma as well as other pathologic eosinophil inflammatory states (e.g., a variety of allergies and skin diseases in which eosinophils have been implicated).

Pseudohalides can be administered by any suitable route, including enteral (e.g., oral administration of a pill or liquid), parenteral injection (e.g., intravenous, subcutaneous, or intramuscular injection), or by nasal administration of an inhalant or aerosol spray. Pseudohalides such as thiocyanate also can be administered as a dietary supplement or by topical application of, for example, a cream or ointment. A preferred route of administration can depend on a variety of factors, such as disease, pharmacokinetic factors, clinician judgment, and therapeutic goals. Pseudohalides such as thiocyanate can be formulated into suitable pharmaceutical compositions as described above by standard techniques.

Any amount of a pseudohalide can be administered provided the dose is effective in treating the inflammation. An effective dose of a pseudohalide depends on many factors including the mode of administration, the severity of the inflammation, and the pharmacodynamic and pharmacokinetic profile of the particular formulation in vivo. The amount of pseudohalide to be administered to a human can be determined by the attending physician taking into account various factors known to modify the action of drugs, including health status, body weight, sex, diet, time and route of administration, other medications, and any other relevant clinical factors. For example, a preparation for inhalation may contain a lower effective dose of the pseudohalide than a preparation for oral administration. Therapeutically effective dosages may be determined by either in vitro or in vivo methods. Effective doses can be, without limitation, less than 0.5 μg, 0.5 μg, 1 μg, 10 μg, 100 μg, 1 mg, 10 mg, 100 mg, 1 g, more than 1 g, or any amount in between the doses stated. Typically, the amount of pseudohalide that is administered is selected to achieve an elevated level (e.g., about 100-250 μM) in serum, other bodily fluids, or the inflamed tissue, without deleterious side effects on the patient.

Treatment of an eosinophil-mediated inflammation can be assessed by determining if eosinophil-mediated inflammation is reduced, i.e., one or more clinical features of the inflammation improve or are stabilized, in the patient following the administration of the pseudohalide. For example, with asthma, clinical features that may improve or stabilize include frequency or length of asthmatic attacks, bronchial constriction, or airway hyperreactivity.

Treatment of inflammation also can include monitoring the level of the psuedohalide or NO in the mammal. Typically, psuedohalide or NO levels are monitored in a biological sample from the mammal, including, without limitation, blood including whole blood, serum, or plasma, mucus, synovial fluid, urine, tissue biopsy, sputum, induced sputum, pericardial fluid, pleural fluid, bronchoalveolar lavage (BAL), or cerebrospinal fluid sample. The level of thiocyanate in the biological sample can be determined using a thiocyanate-reactive reagent. The thiocyanate-reactive reagent can be any compound that reacts with thiocyanate to form a detectable product, including, without limitation, certain transitional metals such as iron (III) or silver. Iron(III) reacts with thiocyanate to form a red complex (FeSCN²⁺) under slightly acidic conditions. NO levels can be measured using the methods described herein, e.g., with a NO specific electrode.

The level of pseudohalide or NO in the biological sample can be compared with a control value to determine if the thiocyanate level or NO level is altered (e.g., increased). The control value can be the level of pseudohalide or NO in the mammal before administration of a pseudohalide. Alternatively, the control value may be representative of the level of pseudohalide or NO in a population of individuals that do not have an inflammatory disease condition or associated complications. Known statistical methods may be used to determine if an increase in thiocyanate or NO is statistically significant.

Articles of Manufacture

The invention also features articles of manufacture such as devices that contain one or more thiocyanate-reactive reagents (e.g., iron (III)). Devices of the invention can be used to identify mammals that have reduced thiocyanate levels, and as such, identify mammals that may benefit from treatment with a pseudohalide. Devices also can include standards or control reagents. For example, the device may contain a known amount of thiocyanate to aid in quantitating the amount of thiocyanate in a biological sample. The device also can include a known amount of the detectable product (FeSCN 2+) for comparative purposes. The device can be a solid substrate such as a membrane, coated paper, nitrocellulose, or microtiter plate. Such devices can be manufactured and sold to organizations involved in managing or providing healthcare.

The invention will be further described in the following examples, which do not limit the scope of the invention described in the claims.

### EXAMPLES

### I. Products of EPO Catalysis

### Example 1

### Source of Reagents

Human EPO, obtained from Dr. Gerald J. Gleich (Mayo Clinic and Research Foundation, Rochester, Minn.), was isolated from granule extracts of purified EO suspensions from patients with hypereosinophilic syndrome as described in Agosti et al. (1987) J Allergy Clin Immunol 79:496-504. Granule extracts were then chromatographed on a Sephadex G-50 column equilibrated with 0.25 M acetate buffer (pH 4.3, 0.15 M NaCl). The fractions eluting with the void volume were then collected and purified to an OD415/280 ratio of >0.9 by chromatography on carboxymethyl Sepharose as described by Carlson et al. (1985) J Immunol 134:1875-1879. SDS-polyacryamide gel electrophoresis was used to confirm homogeneity of the EPO preparation. Two discrete bands (molecular mass of ˜78 and ˜14 kD) that corresponded to the heavy and light chains of EPO, with no contaminating bands, were present. EPO activity was assayed with guaiacol oxidation and converted to international units (the amount of the enzyme that oxidizes 1 μmol of electron donor/min at 25° C. The specific activity of the EPO utilized in the course of these experiments was 133-250 U/mg proteins. Preparations were stored at −70° C. until needed for use and were then maintained at 4° C. wrapped in foil. [¹³C]-labeled (99-atom %) potassium thiocyanate (KSCN) was obtained from Cambridge Isotope Laboratories (Andover, Mass.). Unlabelled KSCN was from Fisher Scientific (Pittsburgh, Pa.). [¹⁴C] SCN⁻ as the potassium salt, specific activity 55.1 mCi/mmol, was from Amersham (Arlington Heights, Ill.). Potassium cyanate was from Baker (Phillipsburg, N.J.). Percoll was from Pharmacia Fine chemicals (Piscataway, N.J.). CD-16 magnetic microbeads were obtained from Miltenyi Biotech Inc (Sunnyvale, Calif.). PMA was from Consolidated Midland Corporation (Brewster, N.Y.). Hank's Buffered Salt solution (HBSS) was obtained from Gibco Laboratories (Grand Island, N.Y.). All other reagents were obtained from Sigma Chemical Co. (St. Louis, Mo.).

### Example 2

### HOSCN is the Major Product of EPO Catalysis in Physiologic Fluids

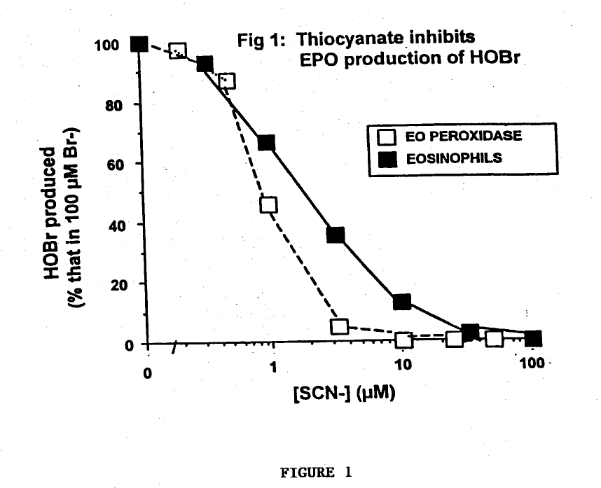

Although SCN⁻ is present in serum at a concentration range that is identical to that of Br⁻ (20-120 μM), SCN⁻ (oxidation potential: −0.77 V) is easier to oxidize than Br⁻ (oxidation potential: −1.07 V). To determine whether the pseudohalide SCN⁻ is the major physiologic substrate for EPO in physiologic fluids, substrate utilization by EPO in buffers of physiologic composition was examined. HOBr production by EOs and EPO in the presence of increasing SCN⁻ concentration was assayed. HOBr production was assayed spectrophotometrically by conversion of fluorescein to bromofluorescein in a buffer containing physiologic Cl⁻ (100 mM) and Br⁻ (100 μM). The results (FIG. 1) show that physiologic concentrations (20-120 μM) of SCN⁻ blocked EPO production of HOBr in the presence of 100 μM Br and 100 mM Cl⁻. SCN⁻ potently inhibits HOBr generation with a 50% inhibitory dose (ID₅₀) of only 1-3 μM, well below the lower limit of its physiologic range (20-100 μM). This was the case both for PMA-activated intact EOs [250,000/mL (open boxes)] and a purified EPO (10 nM)/H₂O₂ (100 μM) system (filled boxes). Even when iodide (I⁻) was present at 100 times its physiologic concentration (100 μM), SCN⁻ again strongly inhibited production of oxidized I⁻ (ID₅₀ 10 μM) as assayed by conversion to monoiodofluorescein. Thus, at physiologically relevant concentrations of SCN⁻ formation of HOCl, HOBr, or HOI was inhibited.

That the product of EPO-catalyzed reactions under such physiological conditions is HOSCN was shown by experiments using [¹⁴C] SCN⁻ in a reagent system with purified EPO and H₂O₂ followed by assaying for the incorporation of the radiolabeled SCN⁻ into stable TCA-precipitable sulfenylthiocyanate-derivatized cysteine residues on bovine serum albumin. SCN⁻ incorporation into proteins in an otherwise halide-free buffer was unaffected by the concomitant presence of physiologic concentrations of Br⁻. Moreover, in human serum spiked with [¹⁴C]SCN⁻, EPO also catalyzed the incorporation of SCN⁻ into serum proteins to an extent compatible with HOSCN being the main or sole product of the reaction. Because the volume of distribution of SCN⁻ included the extracellular space, SCN⁻ is also likely the principal substrate employed by EOs in interstitial spaces where they function. Thus, EPO preferentially oxidizes SCN⁻ in fluids of known physiologic halide composition.

### Example 3

### Temperature- and pH-Dependence of EPO/SCN⁻/H₂O₂ System Oxidants

To characterize the oxidant reaction product(s) generated by the EPO/SCN⁻/H₂O₂ system, thionitrobenzoic acid (TNB)-titratable oxidant stability was examined over time as a function of pH and temperature. Titration of TNB was performed as described in Thomas et al. (1986) J Biol Chem 261:9694-9702. TNB was generated from 5,5′-dithiobis-(2-nitrobenzoic acid) (DTNB) by addition of 2 μL β-mercaptoethanol to 50 mL of a 1 mM DTNB solution. Freshly prepared TNB was calibrated at A412 and combined with EPO reaction mixture to a total volume of 1 mL to determine HOSCN⁻/OSCN⁻ concentration using a molar extinction coefficient of 26,000 M⁻¹ cm⁻¹. Independent studies with reagent OCN⁻ confirmed that this assay does not detect OCN⁻. Under the conditions employed, more total oxidant was initially generated at pH 6.0 than at pH 7.4 (350 μM versus 275 μmol). When subsequently incubated either at 25° C. or 4° C., at pH 7.4 the oxidant half-life was more than six hours at 4° C. and 60 minutes at 25° C. At pH 6.0, the half-life was approximately four hours at 4° C. but less than 30 minutes at 25° C. Thus, oxidant generation by the EPO/SCN⁻/H₂O₂ system is favored at acidic versus physiologic pH; in contrast, oxidant stability is favored at 4° C. and physiologic pH. These stability characteristics are similar to those of the oxidant product of the LPO/SCN⁻/H₂O₂ system.

EPO-catalyzed oxidation of SCN⁻ was performed using conditions similar to those described by Modi et al. using the LPO system. KSCN (2 mM) and EPO (0.4 μM) were added to 0.1 M sodium phosphate buffer at pH 6.0 or 7.4 (total volume 1 mL) and 5 consecutive 250 μM increments of H₂O₂ were added at one minute intervals at room temperature (25° C.), vortexing between additions. One minute after the final bolus addition, 20 μL of catalase (2 mg/mL) was added to consume excess unreacted H₂O₂. Specimens were incubated then at 25° C. or 4° C. and after 0, 30, 90, 120, and 240 minutes, 100 μL aliquots were removed for assay of oxidants by TNB titration.

### Example 4

### NMR Analysis of EPO/[¹³C]SCN⁻/H₂O₂ System Reaction Products and [¹³C] Urea

To enumerate and characterize the major products of the EPO/SCN⁻/H₂O₂ system, [¹³C] NMR analysis of solutions containing [¹³C] SCN⁻ substrate was performed. The [¹³C] SCN⁻ spectra were collected either on a Bruker AMX-600 MHz apparatus using an 8 mm broad-band probe or on a Varian Inova-600 using 5 mm broad-band probe. Spectra were obtained of EPO/[¹³C] SCN⁻/H₂O₂ reaction mixture in 10% D₂O/90% 0.1 M sodium phosphate buffer at either pH 6.0 or 7.4. Reactions were mixed as described in Example 3 to 2 mL (total volume) at appropriate pH at room temperature. One minute after final H₂O₂ bolus addition, 50 μL of catalase (2 mg/mL) was added to reaction tube and reaction mixtures were transferred to ice. Samples were placed in an 8 inch, 8 mm thin-walled NMR sample tube (Wilmad Glass, Buena N.J.). ID spectra were collected using the following parameters, sw=15,009, number of points=32,000, acquisition time=1.066 second, recycle delay of 2 seconds, number of scans either 1500 or 2000. The data were processed using gaussian function 0.099. Chemical shifts were referenced relative to external (CH₃)₄Si. For determination of cyanate spectrum, a 1 M solution of [¹³C]-labeled urea was prepared in PBS buffer (pH 7.4) supplemented with 10% D₂O and incubated for 40 minutes in a water bath at 85° C. to promote equilibration between urea and cyanate. NMR spectra were then obtained. Assay of OCN⁻ by chemical analytic assay (see below) in this preparation confirmed a cyanate concentration of 5 mM.

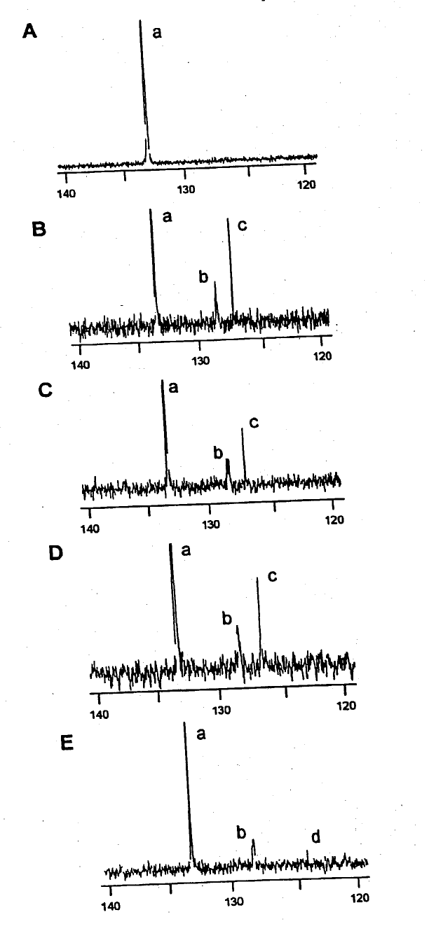

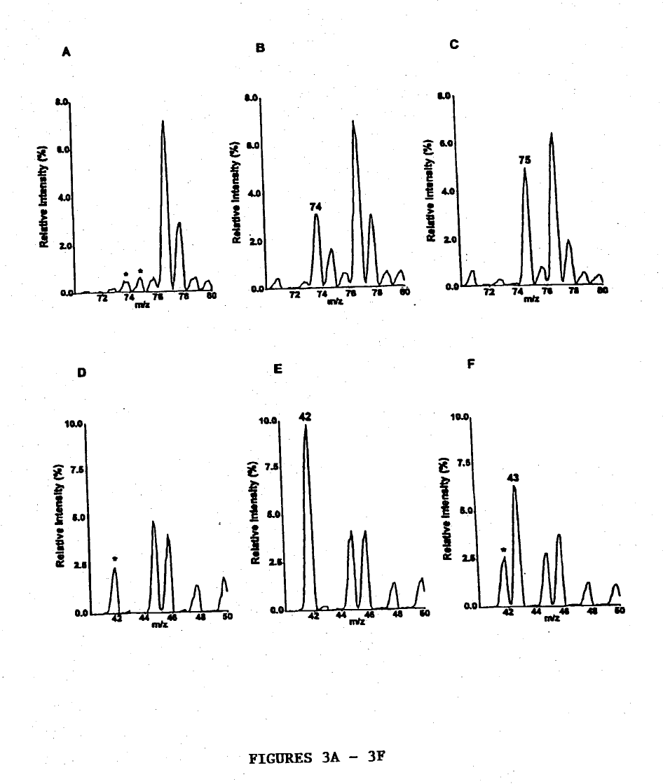

At pH 7.4, spectra accumulated over the first 90 minutes contained the expected large parent [¹³C] SCN⁻ resonance (a) at 133.4 PPM (see FIG. 2). In addition, two new peaks were discernible: (b) at 128.6 PPM along with a more intense resonance (c) at 127.3 PPM (See FIG. 2B). These two latter peaks were absent from the NMR spectrum of [¹³C] SCN⁻ alone or of [¹³C] SCN⁻ in the presence of added H₂O₂ without EPO (FIG. 2A), and are therefore EPO-dependent. Both products were still detectable after incubation overnight at 4° C. at pH 7.4 (FIG. 2C). In contrast, at pH 6.0, although the first spectrum (FIG. 2D) shows peaks b and c, after overnight incubation (FIG. 2E), peak b remained detectable, but peak c has disappeared and a new peak, d, is seen at 124.6 PPM. Thus, peak c has a pH- and temperature-stability profile compatible with that of the TNB-titratible oxidant. In contrast, peak b was relatively stable, at least at 4° C. Peak d, which appearred coincident with the disappearance of peak c, may represent a decomposition product of peak c. Therefore, there are two major initial stable reaction products, i.e. peaks b and c, in the EPO/SCN⁻/H₂O₂ system.

### Example 5

### Electrospray Ionization Mass Spectometry (ESI-MS) and collision-induced dissociation analysis of the EPO/SCN⁻/H₂O₂ system reaction products

To determine the structure of the two major stable products of the EPO/SCN⁻/H₂O₂ system and to rule out the possibility that other products exist, electrospray ionization mass spectometry (ESI-MS) was used to analyze the reaction mixture (FIG. 3). Negative ion ESI-MS was carried out on a triple stage quadrupole mass spectrometer (Perkin Elmer SCIEX API III, Foster City, Calif., USA). The instrument was tuned and calibrated for negative ion operation using polypropylene glycol with the Sciex IonSpray® source.

To confirm that putative reaction products resulted from oxidation of SCN⁻, [¹²C] SCN⁻ and [¹³C] SCN⁻ were used as substrates in the reaction. EPO (0.4 μM) and either [¹³C] SCN⁻ or [₁₂C] SCN⁻ potassium thiocyanate (KSCN) (2 mM) were combined in 10 mM ammonium acetate buffer pH 7.4. Five 200 μM boluses of H₂O₂ were added at room temperature at one minute intervals with mixing and 40 μg/mL of catalase was added to destroy excess H₂O₂. The samples were then placed in Microcon concentrators (Amicon, Mass., USA) with a molecular weight cutoff of 10 kD, centrifuged for 30 minutes (13,000×g at 4° C.) to remove protein, and kept cold until ESI-MS analysis in the negative ion mode. Alternatively, for some experiments samples were filtered through a 0.22 μM exclusion Acrodisc to remove particles and immediately analyzed. Samples were infused into the IonSpray® source at a flow rate of 5-10 μL/min using a syringe pump (Harvard Apparatus Model 22, South Natick, Mass., USA). The various instrumental parameters involved in efficient nebulization and charging of droplets were optimized to the following: spray needle voltage (ISV, −3500 V); interface plate (IN, −650 V); orifice skimmer (OR, −50 to −100 V); Q0 rod offset voltage (−30 V); gas curtain interface (1.2 L/min of N₂ at 60° C.); nebulizer gas (air at 1.5 L/min). Data were acquired in the range of 10 to 100 m/z at a step size of 0.2 AMU and 5 to 10 msec dwell time; 10 to 30 scans were summed over time.

Prior to addition of H₂O₂ to EPO and SCN⁻, a large parent SCN⁻ ion peak with m/z 58 was seen (not shown). FIG. 3A shows the ESI-MS spectrum of an acetate buffer, H₂O₂, and SCN⁻ control solution in the absence of EPO. A large contaminant ion is seen at m/z 77 as are several smaller ions, including two with m/z 74 and 75. When the assay involved the complete EPO/SCN⁻/H₂O₂ system and [¹²C] SCN⁻ (FIG. 3B) an ion with m/z 74 peak was formed. This is consistent with generation of OSCN⁻. When [¹³C] SCN⁻ was substituted for [¹²C] SCN⁻ in an assay using the complete EPO/SCN⁻/H₂O₂ system (FIG. 3C), no significant ion with m/z 74 was generated. That is, ion intensity was the same as in the absence of EPO (FIG. 3A), but there was a large new ion at m/z 75, consistent with formation of [¹³C] OSCN⁻. These results are compatible with OSCN⁻(m/z 74) being one major product of the EPO/SCN⁻/H₂O₂ system.

In scanning the range m/z 10-180 the only other new peak developing in the presence of the EPO/SCN⁻/H₂O₂ system was at m/z=42 (FIGS. 3D and 3E). In the acetate buffer, H₂O₂, and SCN⁻ control in the absence of EPO (FIG. 3D), only a low intensity (i.e., background) ion at m/z 42 was seen. An ion with m/z 42 was generated by the EPO/SCN⁻/H₂O₂ system using [¹²C] SCN⁻, consistent with the formation of [¹²C] OCN⁻. Confirming that this ion arises from SCN⁻, substitution of [¹³C] SCN⁻ for [¹²C] SCN⁻ yielded a prominent new ion with m/z 43 instead of 42 (FIG. 3F). Under these conditions, the m/z 42 ion intensity is the same as in the control lacking EPO (FIG. 3D) indicating that this background m/z 42 ion is likely a contaminant in our buffer system. These results suggest that OCN⁻ (m/z 42) is the second major product of the EPO/SCN⁻/H₂O₂ system.

To characterize these compounds further, collision-induced dissociation tandem mass spectrometric analysis (MS/MS) was used. MS/MS studies were performed on a Micromass Quatro II triple quadruple mass spectrometer (Altrincham, U.K.). Collision-induced mass spectra were determined in the negative ion mode by direct infusion of reaction products (formed by the EPO/SCN⁻/H₂O₂ system as described for the ESI/MS studies above) at a flow rate of 10 μL/min (Harvard Apparatus pump). Mass spectra analysis was performed with a cone potential of 20 eV, collision energy of 30 eV, collision gas (Ar) cell at 1.7×10⁻³ mbar, source temperature 70° C., capillary 3500 V, high voltage lens 690 V, skimmer offset of 5 V.

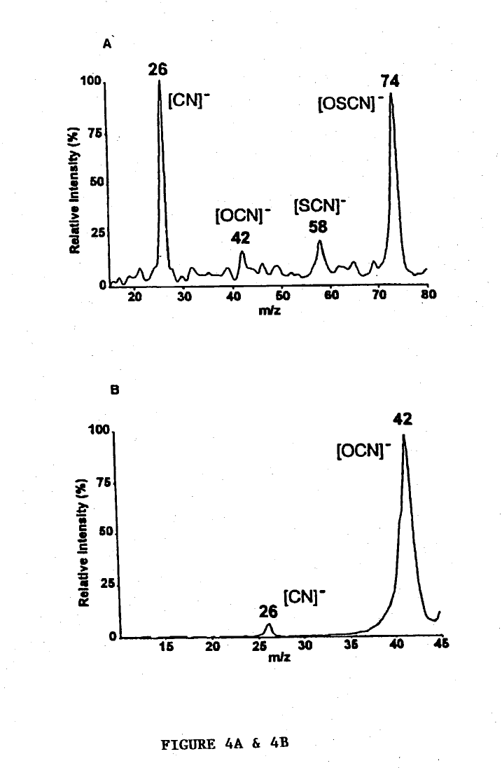

As shown in FIG. 4A, when [¹²C] SCN⁻ was used as the substrate, the parent ion with m/z 74 fragmented to yield a predominant daughter ion of m/z 26 (compatible with CN⁻), as well as less abundant ions at m/z 58 (compatible with SCN⁻) and m/z 42 (compatible with OCN⁻). Similarly, the m/z 42 ion also fragmented, though less extensively, to yield a daughter ion of m/z 26 (FIG. 4B). In parallel MS/MS studies of ions at m/z 75 and at m/z 43 arising from substitution of [¹³C] SCN⁻ for [²C] SCN⁻, both yielded daughter ions consistent with these assignments. Collectively, these data strongly suggest that the ions representing the major catalysis products of the EPO/SCN⁻/H₂O₂ system are attributable to OSCN⁻ and OCN⁻.

### Example 6

### Analytic Quantitation of Cyanate

OCN⁻ was assayed using the method of Guilloton & Karst (see Guilloton et al. (1985) Analytical Biochemistry 149:219-295). Briefly, 250 mL of a freshly prepared 10 mM solution of 2-aminobenzoic acid (anthranilic acid) prepared in 50 mM sodium phosphate buffer at pH 4.4 was incubated with 250 μL of the EPO system reaction mixture for 10 minutes at 40° C. The mixture was then added to 500 μL 10N HCl, then boiled for one minute, allowed to cool to room temperature and assayed spectrophotometrically at 310 nm with a molar extinction coefficient of 3.56 mM⁻¹ cm⁻¹. A KOCN standard curve was used to determine specific concentration. This assay does not detect HOSCN/OSCN⁻ because EPO system reaction mixtures contained the same amount of cyanate before and immediately after selective titration of HOSCN/OSCN⁻ by addition of dithiothreitol.

### Example 7

### Production of Oxidants and Cyanate by the EPO/SCN⁻/H₂O₂ System: Time Course and Peroxidase Dependence

To determine the relative time courses of HOSCN and cyanate generation, H₂O₂ was added to a reaction mixture containing phosphate buffered saline (PBS) with 1 mM KSCN, pH 7.4, and 0.4 μM EPO. H₂O₂ was added by continuous infusion at 50 μM/minute for 20 minutes to simulate the continuous generation of H₂O₂ by the respiratory burst of an activated EO. An aliquot was (i) removed from the reaction mixture prior to and at various time intervals after initiating addition of H₂O₂, (ii) supplemented with 40 μg/mL catalase to consume unreacted H₂O₂, and (iii) placed on ice until subsequent assay of oxidants and cyanate as described. To establish peroxidasedependence generation of oxidant and cyanate by the EPO/SCN⁻/H₂O₂ system, the effect of either omitting EPO or adding the EPO inhibitor azide (1 mM) was assayed. The assay consisted of adding five consecutive increments of 100 μM H₂O₂ at one minute intervals to PBS (pH 7.4), 0.4 μM EPO, and 1 mM KSCN. The reaction was terminated by adding 100 μg/mL catalase and placing the specimen on ice prior to assaying oxidants and cyanate.

### Example 8

### Purification of Peripheral Blood Eosinophils

The anti-CD-16 immunomagnetic bead cell sorting system (MACS, Miltenyi Biotec Inc.) was used as described by Ide et al. (1994) J Immunol Methods 168:187-196, with minor modifications. About 60 mL of citrated blood from eosinophilic donors (n=3) undergoing interleukin-2 immunotherapy were mixed with 30 mL of 6% Hetastarch in a 0.9% NaCl solution (Abbott Labs, IL). Erythrocytes were allowed to sediment for 45 minutes at room temperature and the leukocyte-rich fraction was collected. This fraction was diluted with an equal volume of PBS (137 mM NaCl, 3 mM KCl, 4.2 mM sodium phosphate and 1.5 mM potassium phosphate, pH 7.4) supplemented with 2% fetal bovine serum. The resulting sample was layered atop a half-volume of isotonic Percoll (density 1.082 g/mL) in 50 mL conical tubes, and centrifuged for 30 minutes at 1000×g and 4° C. The supernatant and mononuclear cells at the interface were carefully aspirated and the inside wall of the tube wiped with sterile cotton tip applicators to remove residual mononuclear cells. The pellet of granulocytes and remaining erythrocytes were subjected to a hypotonic lysis by exposure to 20 mL of ice-cold sterile water for 30 seconds. The granulocytes were rescued with 20 mL of a 2×isotonic buffer (40 mM HEPES, 10 mM KCl, 10 mM D-glucose, 280 mM NaCl, pH 7.4) and pelleted. The lysis was repeated if erythrocytes remained. The specimen was then incubated with anti-CD-16-coupled immunomagnetic beads and processed as described in Hansel et al. (1990) J Immunol Methods 127:153-160. The resulting preparations all contained ≧98% EOs.

### Example 9

### EO Production of Oxidants and Cyanate

Purified EOs were washed, suspended at 4×10⁶/mL in a modified Hank's balanced salt solution composed of 5.3 mM KCl, 138 mM NaCl, 0.4 mM potassium phosphate, 5.3 mM sodium phosphate, 5.5 mM D-glucose, 1.3 mM CaCl₂, and 0.5 mM MgCl₂, pH 7.4 supplemented with 1 mM KSCN. EOs were divided into three groups that were supplemented with (i) nothing (control), (ii)1 μg/mL phorbol 12-myristate 13-acetate (PMA), or (iii) 1 μg/mL PMA and 5 mM azide. EOs were then incubated at 37° C. for 60 minutes in capless 5 mL round-bottom tubes. The tubes were vortexed every 15 minutes to aid oxygenation. After the incubation, the samples were centrifuged for 10 minutes, 1000×g at 4° C. The supernatants were placed in Microcon concentrators (Amicon, Mass.) with membranes of 3000 Dalton molecular weight cut off and centrifuged for 30 minutes, 13,000×g at 4° C. to remove degranulated proteins. The filtrates were assayed for HOSCN and cyanate quantities.

### Example 10

### OSCN⁻/OCN⁻ Effects Upon Red Blood Cell (RBC) Glutathione and Enzymes

Human RBCs were suspended at a hematocrit of 2% in PBS (pH 7.4) supplemented with increasing concentrations of OSCN⁻/OCN⁻ generated as above by the EPO/SCN⁻/H₂O₂ system. After 30 minutes, cells were pelleted by centrifugation at 2000×g for 3 minutes and washed three times in ice-cold HEPES/Hanks' (H & H) buffer. Under these conditions, hemolysis was detected only at ≧200 μM OSCN⁻/OCN⁻ by spectrophometric assay of supernatant hemoglobin or methhemoglobin. RBC lysates and membranes were then prepared by hypotonic lysis and assayed for glutathione and glyceraldehyde-3-phosphate dehydrogenase (GAPDH), glutathione-S-transferase (GST), and lactate dehydrogenase (LDH) activity as described by Beutler (see Beutler (1974) Red Cell Metabolism A Manual of Biochemical Methods Grune and Stratton, Inc., Orlando, Fla.) and for ATPase activity as described below. Hemolysates were incubated for an additional 15 minute with or without 10 mM DTT and reassayed for GAPDH, GST, and ATPases.

### Example 11

### Comparative Oxidant Inactivation of RBC ATPase Activity

Human RBC membranes were prepared by hypotonic lysis. RBC membranes were suspended at 0.2 mg protein/mL in membrane buffer. Membrane buffer contained 20 mM Hepes, 5.5 mM NaCl, with 1 g/L D-glucose, 0.10 g/L MgCl₂.6H₂O, 0.10 g/L MgSO₄.7H₂O, and 0.4 g/L KCl, pH 7.4 supplemented with increasing concentrations of H₂O₂, HOCL, HOBr, or OSCN⁻/OCN⁻. HOCl was diluted from concentrated HOCl (Sigma). HOBr was prepared by adding excess Br⁻ to stock HOCl as described in Slungaard et al. (1991) J Exp Med 173:117-126. OSCN⁻/OCN⁻ was prepared by EPO as described above. RBC membranes were incubated with the oxidants for 15 minutes at 37° C. then pelleted and washed three times in ATPase buffer without oxidant. Total ATPase activity, i.e., Ca²⁺-, Mg²⁺-, calmodulin- and Na⁺/K⁺-dependent ATPases, was assayed using 0.2 mg protein RBC membrane aliquots suspended in 500 μL ATPase buffer. ATPase buffer contained 90 mM histidine, 90 mM imidazole, 15 mM MgCl₂, 400 mM NaCl₂, 0.5 mM EGTA, 75 mM KCl, 1 mM Ca Cl₂, 300 nM calmodulin, pH 7.4. ATPase activity was assayed by adding 3 mM ATP to RBC membrane aliquots, incubating for 10 minute at 37° C., then quenching phosphate release by adding 1000 μL of 1% ascorbate in 10% trichloroacetic acid. Phosphate release was quantitated by adding 250 μL of 1% ammonium molybdate tetrahydrate, incubating 5 minutes, then adding 500 μL of 2% sodium citrate dihydrate and 2% sodium arsenite in 2% acetic acid. The resulting solution was assayed with a spectrophotometer at 700 nm and amounts of phosphate calculated from a standard curve generated using H₂ KPO₄. Values were corrected for both spontaneous membrane phosphate release (i.e., membranes without ATP added) and spontaneous ATP hydrolysis (i.e., ATP in the absence of membranes).

### Example 12

### PAGE Gels of GST Exposed to [¹⁴C] OSCN⁻/OCN⁻

About 10 μg of human GST-π was exposed to 200 μM [¹⁴C] OSCN⁻/OCN⁻ (generated using [¹⁴C] SCN⁻ in the EPO/SCN⁻/H₂O₂ system described above) or H & H buffer. Samples were suspended in Laemmli buffer with or without β-mercaptoethanol (BME) and separated on a 3-15% SDS/PAGE gel. Gels were stained with Coomassie Blue and autoradiograms developed by fluorographically enhanced exposure of X-ray film (Kodak X-AR).

### Example 13

### Quantitative Detection of OSCN⁻ and Cyanate as Major Products of Both the EPO/SCN⁻/H₂O₂ System and Activated Human EOs: Kinetic relationship between OSCN⁻ and OCN⁻ Generation

Detection of an ion with m/z compatible with OCN⁻ by mass spectrometry could represent an artifact of OSCN⁻ fragmentation during the ionization process. Therefore, to ascertain whether OCN⁻ is present in the EPO/SCN⁻/H₂O₂ system reaction product prior to mass spectrometry analysis, a well-characterized analytical assay based upon the reaction of OCN⁻ with 2-aminobenzoic acid and subsequent cyclization in acid conditions to form 2,4 (1H,3H)-quinazolinedione, which is detected at 310 nm (see Guilloton et al. (1985) Analytical Biochemistry 149:219-295) was used. The reaction mixture was titrated with dithiothreitol to show that that this assay for OCN⁻ did not also detect HOSCN. The absence of HOSCN was confirmed by ESI-MS. In addition, the OCN⁻ assay detected similar amounts of OCN⁻ before and after the selective depletion of HOSCN.

Using this assay, the EPO/SCN⁻/H₂O₂ system was found to generate large amounts of cyanate with a time course that lags behind that of OSCN⁻. Simultaneous determinations of oxidant (as TNB-titratible material) and OCN⁻ (as determined in the aminobenzoic acid reaction) at various time-points during the progression of the EPO/SCN⁻/H₂O₂ reaction was observed. H₂O₂ was continuously infused at 50 μM/minute for 20 minutes, simulating the continuous generation of H₂O₂ by the respiratory burst of an activated EO. Aliquots were removed from the reaction mixture at one-minute intervals and catalase was added to destroy excess unreacted H₂O₂ prior to assay of TNB-reactive substances and cyanate. OCN⁻ generation initially “lags” behind that of HOSCN for 5 minutes. HOSCN production plateaus at about 175 μM while cyanate accumulation continued only so long as H₂O₂ infusion continued (i.e., 20 minutes) then abruptly ceased thereafter. This plateauing of HOSCN levels during H₂O₂ infusion reflected a balance between rapid generation by the EPO and consumption by some secondary reaction, because addition of the EPO inhibitor azide at this stage of the progression curve caused an abrupt collapse of HOSCN levels. Taken together, these data suggest that HOSCN is the initial product of the EPO/SCN⁻/H₂O₂ system and that OCN⁻ results from a subsequent reaction of HOSCN—e.g., reaction with excess H₂O₂, reaction with a second molecule of HOSCN, or spontaneous decomposition.

To determine which of the NMR resonance peaks (i.e., b or c) detected in FIG. 2 represents OCN⁻, the NMR spectrum of [¹³C] urea was analyzed. Although [³C] OCN⁻ is not commercially available, urea exists in equilibrium with small (approximately 0.5% at physiologic pH) amounts of cyanate. The NMR spectrum of [¹³C] urea at pH 7.4 shows the expected large main urea peak at 162.5 PPM as well as a smaller OCN⁻ peak at 128.6 PPM. The shift of this peak is indistinguishable from that of peak b in FIG. 2, supporting its identification as OCN⁻. Since sulfur is less electron withdrawing than oxygen, the upfield resonance (peak c in FIG. 2) is likely [¹³C] OSCN⁻.

To rule out non-enzymatic oxidation of SCN⁻ by H₂O₂ as the basis for OCN⁻ generation by the EPO/SCN⁻/H₂O₂ system EPO was omitted or, alternatively, EPO catalytic activity was blocked with the potent inhibitor azide. Under both conditions, generation of both HOSCN and OCN⁻ were nearly completely blocked suggesting that generation of OCN⁻ as well as HOSCN depended upon the catalytic action of EPO.

As a gauge of the potential physiologic relevance of the above findings—all made with a purified reagent system—intact, activated human EOs were examined to determine whether HOSCN and OCN⁻ are generated. Peripheral blood EOs were isolated from cancer patients undergoing experimental therapy with subcutaneous injections of interleukin-2, a circumstance known to be associated with the secondary elevation of blood interleukin-5 with consequent eosinophilia and activation of circulating EOs. EOs isolated from such patients spontaneously generated low but detectable amounts of HOSCN and OCN⁻. When maximally stimulated by addition of phorbol myristate acetate, EOs generated greatly increased and roughly equimolar amounts of both HOSCN and OCN⁻. Addition of 5 mM azide severely attenuated phorbol myristate acetate-stimulated generation of both products, though HOSCN more so than OCN⁻. Thus, activated human EOs generate both HOSCN and OCN⁻ by an EPO-dependent mechanism.

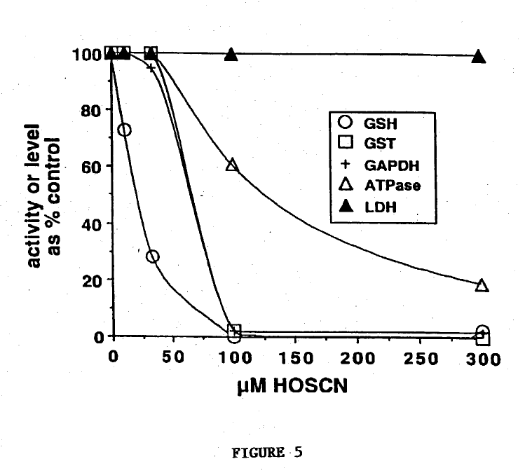

To determine whether products of the EPO/SCN⁻/H₂O₂ system react almost exclusively with SH groups to inflict SH-based toxicity, intact human RBCs were exposed to increasing concentrations of OSCN⁻/OCN⁻ generated by the EPO/SCN⁻/H₂O₂ system. Intracellular levels of the SH-containing oxidant scavenger glutathione were assayed. In addition, the activities of three enzymes, GST, GAPDH, and membrane ATPases, which are known to be vulnerable to SH-oxidation-based inactivation, as well as LDH which is not prone to such inactivation were assayed. Exposure of RBCs to OSCN⁻/OCN⁻ under these conditions produced no detectable hemolysis. As illustrated in FIG. 5, with increasing concentrations of OSCN⁻/OCN⁻, glutathione was first depleted, followed by inactivation of GST and GAPDH, then membrane ATPases. LDH activity was completely unaffected. This inactivation of GST, GAPDH, and ATPase activity was based on reversible SH oxidation because subsequent treatment of lysates and membranes prepared from RBCs exposed to 100 μM OSCN⁻/OCN⁻ with 10 mM dithiothreitol (DTT), a SH reducing agent, completely restored the activities of all three enzymes. Therefore, these findings suggest that EPO/SCN⁻/H₂O₂ products diffuse through intact RBC membranes to oxidize intracellular SH-containing compounds.

To determine whether the potential specificity of OSCN⁻/OCN⁻ for SH reactivity, more effectively mediates SH-based toxicity than the potent bleaching oxidants HOCl and HOBr, the capacity of these oxidants as well as H₂O₂ to inactivate ATPases in isolated RBC membranes was compared. Membrane preparations rather than intact RBC were used to allow all oxidants equal access to intra- and extracellular membrane components. In this model OSCN⁻/OCN⁻ inhibited RBC ATPases more effectively than in intact RBCs with an ID₅₀ of 2 μM, 1/10^{th} that of HOCl and HOBr; H₂O₂ failed to inactivate ATPases even at 1 mM. Thus, the failure of OSCN⁻/OCN⁻ to react indiscriminantly with a wide array of other membrane components allows it to function more effectively than do more potent oxidants as a SH-based metabolic inhibitor.

To examine the structural basis of SH-dependent inactivation of GST by OSCN⁻/OCN⁻, human GST-π, the isoform found in RBCs, was exposed to [¹⁴C] OSCN⁻/OCN⁻. Proteins were then analyzed by SDS/PAGE for electrophoretic mobility and [¹⁴C] incorporation under reducing and non-reducing conditions. SDS/PAGE results indicate that GST not exposed to [¹⁴C] OSCN⁻/OCN⁻ ran as a single band with a mobility of 23 kD under reducing conditions. In contrast, under non-reducing conditions an additional faint band of higher mobility, presumably representing a species containing intramolecular disulfide bonds, was evident. After exposure to [¹⁴C] OSCN⁻/OCN⁻, under nonreducing conditions, the 23 kD was reduced in intensity, the higher mobility band increased, and an additional faint band of ca. 48 kD was also seen. That this last band represents disulfide-bonded intermolecular dimers is shown by the fact that it, along with the high mobility band, disappear upon reduction. Autoradiograms showed heavy, nearly completely SH reductant-reversible binding of [¹⁴C] into all three of these bands as well as a faint band of even higher mobility. Thus, exposure of GST to [¹⁴C] OSCN⁻/OCN⁻ caused intra- and intermolecular disulfide bond formation, as well as SH reductant-reversible bonding of a [¹⁴C]-containing moiety.

### II. The Cytotoxicity of EPO-Based Oxidation Products is Hypobromide>NO₂.>>HOSCN

### Example 1

### HOBr is Highly Toxic for Host Cells

Because Br⁻ is a potential substrate for EPO, the toxicity of the resulting oxidant, HOBr, for host human cells was examined. EPO is detectable on the endocardial surfaces in the hearts of patients with hypereosinophilic heart disease. When purified EPO was bound to the surface of cultured endothelial cells, addition of micromolar concentrations of H₂O₂ in the presence of physiologic concentration of Br⁻ (100 μM) led to destruction of endothelial cell monolayers. Binding of EPO to the vasculature and the left ventricular cavity in an isolated perfused rat heart model followed by subsequent perfusion with physiologic concentrations of Br⁻ and 1 μM H₂O₂ led to an abrupt onset of congestive heart failure. Thus, HOBr, like HOCl, is highly toxic for host tissue.

### Example 2

### HOSCN is Markedly Less Toxic Than HOBr for Endothelial Cells and Working Rat Hearts

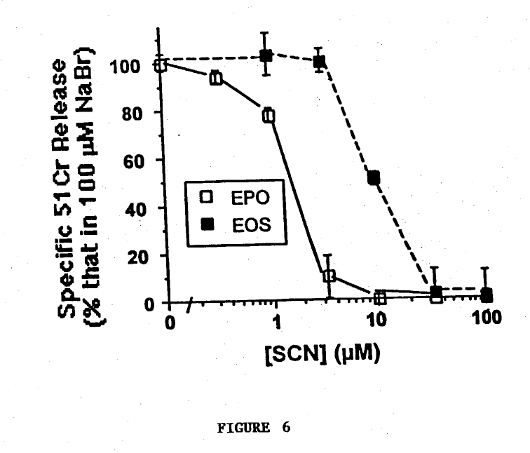

As shown in FIG. 6, when ⁵¹Cr-labeled endothelial cells were exposed to intact EOs, or purified EPO and H₂O₂, Br⁻ dependent toxicity was nearly obliterated by the presence of even subphysiologic amounts of SCN⁻. The apparent decreased sensitivity of EO toxicity to SCN⁻ inhibition relative to EPO is attributable to consumption by EOs (1,250,000/mL) of available SCN⁻ and subsequent oxidation of Br⁻ only after SCN⁻ had been depleted. This result was confirmed in the isolated working heart model, where 100 μM and 10 μM SCN⁻ completely blocked the development of acute congestive heart failure otherwise seen in the presence of 100 μM Br⁻.

### Example 3

### Comparative Toxicity of HOSCN and HOBr for Mammalian Cells and Schistosomules

To determine how generation of HOSCN subserves the physiological function of EOs, i.e., to destroy tissue-invasive parasites, the following experiment was performed. Reagent grade HOSCN was generated by (i) adding human EPO (10 μg/mL) and 1 mM NaSCN to Hank's buffer, (ii) adding five 100 μM boluses of H₂O₂ at 3 minute intervals, (iii) adding 20 μg/mL of catalase to consume unreacted H₂O₂, and (iv) removing protein by filtration through a PM-10 membrane (Amicon). HOSCN was then assayed by TNB titration. Mammalian cells used included cultured human and porcine aortic endothelium, rat cardiac myocytes, P338D1 murine lymphoma, and RBCs. Freshly transformed S. mansoni schistosomules (obtained from James Kazura at Case Western University) were also used. Both were exposed to reagent HOSCN for two hours, then assayed for cytotoxicity by (i) ⁵¹Cr release, (ii) hemolysis (mammalian cells) or (iii) light microscopy observations of granularity, lack of motion, and methylene blue dye exclusion (somules). Results in Table 1 indicate that HOSCN killed both schistosomules and host cells. Whereas HOCl and HOBr were equally toxic for host cells and somules, HOSCN was 10-15 times more toxic for the parasite. These findings suggest that HOSCN is an oxidant toxin with relative specificity for pathogens over host mammalian tissue.

TABLE 1
Comparative toxicity of hypohalous acids for mammalian cells and
schistosomes

| Oxidant | LD₅₀ for mammalian cells | LD₅₀ for schistosomules |
|---|---|---|
| HOCI | 10-33 μM | 10 μM |
| HOBr | 10-33 μM | 10 μM |
| HOSCN | 200-300 μM | 20 μM |

In contrast to highly reactive surface-acting oxidants like HOCl, the cytotoxicity of HOSCN reflects oxidative modification of functionally important SH residues in critical intracellular proteins. Furthermore, the relatively low reactivity of HOSCN confers the capacity to function with greater specificity, as a SH-reactive metabolic poison for parasites. This specificity might permit HOSCN to exploit metabolic differences between parasitic pathogens and host tissues to effect selective toxicity.

### Example 4

### Determination of the Threshold of Lysis for SCN⁻, Br⁻ and NO₂⁻-Dependent EO Peroxidation Oxidants

To determine the threshold of cell lysis for SCN⁻, Br⁻ and NO₂⁻-dependent EO peroxidation oxidants, two cell lines are used: the BEAS-B2 cell line and the small airway epithelial cell line (SAEC) from Clonetics. The BEAS-B2 cell line is derived from normal human bronchial epithelium and transformed by infection with an adenovirus 12-SV40 virus hybrid. It is obtained from the ATCC and is maintained in serum-free LHC-9 medium. SAEC are primary cultures derived from bronchoalveolar lavage (BAL) specimens from volunteer normal humans, and differentiate during passage to Clara cells or ciliated epithelial cells. These are maintained in serum-free SAEC basal medium (CCMD 160; Clonetics) supplemented with growth factors (bovine pituitary extract, hydrocortisone, human recombinant epidermal growth factor, epinephrine transferrin, insulin, retinoic acid, triiodothyronine, gentamycin, and amphotericin) with fatty acid-free bovine serum albumin. These cells are passaged four to six times at most.

Using the following protocol, BEAS-B2 and SAEC are exposed to increasing concentrations of the relevant halide-specific oxidants by regulating the amount of hydrogen peroxide added to each monolayer (0-500 μM). ⁵¹Cr release, Trypan Blue dye exclusion, and amount of release of the intracellular enzyme LDH into the supernatant are used to determine the threshold for cell lysis.

To expose cells to oxidants, supernatant media of freshly confluent monolayers of BEAS-B2 or SAEC are aspirated, then monolayers are layered with HEPES/Hanks' buffer (H & H) supplemented with 1 mM Br⁻, SCN⁻, or NO₂⁻. Purified human EPO (100 nM) and increasing concentrations of hydrogen peroxide are added, cells are incubated for 4 hours at 37° C., then 20 μg/ml of catalase are added to consume any unreacted hydrogen peroxide. Supernatants are aspirated and saved for ATP and LDH determination. Cells are rapidly trypsinized and recovered in single cell suspension for subsequent analysis of Trypan blue dye exclusion, propidium iodide exclusion, and further assays of cellular integrity as described. Alternately, for assay of more subtle aspects of cell dysfunction such as swelling, cells are first trypsinized into single cell suspension, then exposed to oxidant using the protocol described above.

### Example 5

### Determination of Membrane Versus Intracellular Oxidation Stress Induced by EPO-Dependent Oxidants

Trypsinized single cell suspensions of the appropriate cell lines are exposed to halide-specific oxidant stress at sublytic concentrations for one hour as described above. The following assays are then performed.

Cell volume is determined by staining with propidium iodide. Cell size distribution is analyzed using FACS analysis. A 75 μM orifice previously calibrated using various diameter microbeads (Coulter, Hialeah, Fla.) (see Schraufstätter et al. (1990) Clin Invest 85:554-562) is used.

Oxidation status of SH groups on extracellular proteins are assayed using the non-permeant probe iodoacetylated R-phycoerythrin (IPC) (Molecular Probes, Eugene, Oreg., MW=240,000) and subsequent FACS analysis using rhodamine filters. To subtract background fluorescent, cells are exposed to 16 mM β-mercaptoethanol before the addition of IPE, which completely prevents labeling of cells. Total cellular protein SH status is assayed by exposing aliquots of 2×10⁶ cells to oxidants for 1 hour. Cellular protein is precipitated with 400 μL 50% TCA, samples are left on ice for 10 minute, microfuged for 1 minute, and washed twice with 7% TCA. For reduced sulfhydryl determination, the precipitate is resuspended by sonication in 2 M guanidine thiocyanate, 500 mM Tris, 10 mM EDTA, pH 7.6, 100 μM dithionitrobenzoic acid (DTNB; Sigma Chemical Co., St. Louis, Mo.). Sulfhydryls reduce DTNB forming yellow colored TNB. Alternatively, the precipitate is resuspensed in 2 M guanidine thiocyanate, 50 μM glycine, 100 mM sodium sulfite, 3 mM EDTA, 100 μM 2-nitro-5-thiosulfobenzoate (NTSB), pH 9.5. The sulfite cleaves disulfide bonds, such that the sum of-SHs and disulfides is measured by reduction of NTSB. The samples are then be incubated for 25 minutes at room temperature, and the OD₄₁₅/₅₅₀ nm is measured on Titertek plates from 200-μL aliquots (Autoreader, EL 310; Biotek, Inc., Burlington, Vt.). Molarities are calculated assuming an extinction coefficient of e₄₁₅=12,200 m⁻¹ cm⁻¹ for TNB. Carbonyl carbon formation as is assayed as a measure of irreversible oxidative degradation of proteins by the formation of protein hydrazone derivatives as described by Starke et al. (see Starke et al. (1987) FASEB J 1:36-39).

Intracellular glutathione concentration is assayed by DTNB titration of perchloric acid extracts of cells as described in Bellomo et al. (1985) Hepatology 5:876-882.

Cytosol and membrane preparations are prepared by nitrogen cavitation and differential centrifugation after suspension in 0.34 mol sucrose, 10 mmol Hepes, 1 mmol EGTA, 0.1 mmol MgCl and 1 mmol ATP as described by Schraufstetter et al. (see Schraufstätter et al. (1990) Clin Invest 85:554-562). Membrane fraction is assayed for ATPase activity by molybdate complex quantification (see Baginski et al. (1967) Clinica Chimica Acta 15:155) of free phosphate liberation in the presence or absence of ouabain to assay sodium potassium ATPase. Cytosol fractions are assayed for ATP and GST by CDNB conjugation (see Sonenshein (1997) Semin Canc Biol 8:113-119) as well as GAPDH. Assay of cytosolic fractions after exposure to 10 mol DTT establishes whether inactivation is due to a sulfhydryl reversible oxidative interaction.

Lipid peroxidation is assayed as 8-ispoprostanes (8-epi and 8-iso PGF_{2α}) using a commercially available enzyme immunoassay kit (Cayman Chemical, Ann Arbor, Mich.) following lipid extraction and addition of hyrdroxybutyrate and deferoxamine to avoid artifactual generation of lipid peroxidation during the course of the assay.

### Example 6

### Protein Structural Analysis of GAPDH, a Model Intracellular SH-Dependent Enzyme

The following experiment is performed to analyze the mechanistic basis for the inactivation of GAPDH after exposure to the three potential EPO-catalyzed oxidants. HOSCN can modify thiols (SH) in at least two different ways. It can react with two SHs to yield a disulfide (intra- or inter-molecular) [HOSCN+2 RSH=RSSR+SCN⁻+H⁺] or with a single SH to form a stable sufenyl thiocyanate [RSH+HOSCN=R−S−SCN+H₂O]. HOCl and HOBr cannot form stable sufenyl SH derivatives as does HOSCN (RS−SCN=soft-soft), but instead form stable chloramines (RNHCl=hard-hard).

The first level of analysis employs SDS PAGE separation of membrane and cytosolic proteins from cells previously exposed to either buffer or HOSCN, then exposed to iodo [¹⁴C] acetic acid to label all remaining SH groups. Scanning video densitometry that measures HOSCN⁻ dependent decrements in free SHs in specific proteins (see Brodie et al. (1990) Arch Biochem Biophys 276:212-218) is used to quantitate bands appearing on fluorographically enhanced autoradiograms of these gels. Addition of DTT to the cell extraction buffer prior to labeling with iodo [¹⁴C] acetic acid establishes the SH-specificity and reversibility of target protein SH oxidation by HOSCN.

The second level of analysis is to determine whether [¹⁴C] HOSCN forms covalent sulfenyl thiocyanate (R—S—SCN) derivatives of GAPDH thiols, i.e, whether exposure of GAPDH to this oxidant results in incorporation of [¹⁴C] SCN⁻ in a SH-reductant reversible manner. Cells are exposed to [¹⁴C] HOSCN, cellular extracts and cell fractions are separated on SDS PAGE gels under both non-reducing and reducing conditions. The separated proteins are detected using fluorographically enhanced autoradiograms.

### Example 7

### Comparison of the Ability of HOSCN, NO₂., and HOBr to Vause Necrotic Versus Apoptotic Cell Death in Small airway Epithelial Cells

BEAS-B2 and SAEC are exposed to respective oxidants as described in Example 4 for 1 hour. Cells are then either trypsinized for analysis or “rescued” with either simple H & H buffer, or complete media, and analyzed periodically over the ensuing 48 hours to examine whether necrotic or apoptotic cell death ensues. Apoptosis is assayed in the following ways.

Morphological assessment is performed using Hoechst 3342 and propidium iodide nuclear staining and fluorescence microscopy (see Volkl et al. (1992) Fortschr Med 110:346-350). Cells are assessed by a blinded observer and classified in the following manner. Live cells are those with normal nuclei, blue chromatin, and organized structure. Membrane intact apoptotic cells have bright blue chromatin that is highly condensed, marginated or fragmented. Necrotic cells have red enlarged nuclei with smooth normal structure. Membrane-permeable apoptotic cells have bright red chromatin that is highly condensed or fragmented. Pyknotic necrotic cells have dense, bright red, slightly condensed nuclei that are sometimes divided into 2 or 3 spheres.

Conventional agarose gel electrophoresis is used for detection of nucleosomal DNA fragmentation. Total cellular DNA is extracted and applied to 2% agarose gels. Electrophoresis is performed using 1×TBE for 12-15 hours at 30 volts. HAE-III-digested Φ-X174 (New England Biolabs, Beverly, Mass.) is used as molecular weight standard.

Annexin-FITC binding analysis is performed on flow cytometry using the R&D Systems apoptosis detection kit employing FITC-conjugated annexin in a 530/30 nm band pass filter and PI measured at 610 nm.

Apoptosis is also assessed using terminal deoxyribonucleotidyl transferase-mediated dUTP-biotin nick end labeling assay (TUNEL).

There can be dramatic differences in the patterns of oxidant stress and cell damage caused by HOSCN, NO₂⁻., and HOBr. HOSCN can be, on a molar basis, much less toxic for human airway cells and, in addition, there can be differences in the patterns of intracellular versus extracellular oxidative stress. Specifically, HOSCN penetrates intact cells and produces intracellular depletion of glutathione followed by, at very high concentrations, inhibition of sulfhydryl-dependent enzymes including GAPDH. In contrast, NO₂⁻. and HOBr causes cell death specifically by lipid peroxidation (assayed as TBARS) and protein membrane oxidation (external membrane SH oxidation), respectively, leading to direct breaching of the membrane integrity prior to producing any sort of intracellular oxidative damage (intracellular SH oxidation and carbonyl carbon formation). This has important implications for the pathogenesis of asthma and its variability in various populations and suggests potential therapeutic strategies. For example, the noted increased severity of asthma in certain minority populations may reflect genetic variation in the activity of key antioxidant enzymes, particular glucose-6-phosphate dehydrogenase, a well-known genetic variant common in African-Americans, Mediterraneans and other populations. If NO₂. oxidation produces primarily lipid peroxidation stress, then other antioxidant strategies such as vitamin E might prove efficacious. Both NO₂. and HOBr are scavenged readily (see Wu et al. (1999) J Biol Chem 274:25933-25944) by the abundant plasmatic antioxidant, ascorbic acid, but HOSCN is not (see Chesney et al. (1991) Anal Biochem 196:262-266).

HOSCN, if it produces cell death, does so through the apoptotic pathway, in contrast to NO₂. and HOBr, which produce frank cell lysis. This is based on the finding that HOSCN penetrates intact membranes to oxidize intracellular proteins, and intracellular oxidative reactions can promote apoptosis as preliminary TUNEL data indicates. This further supports the rationale for avoiding SCN⁻ depletion and consequent enhancement of NO₂⁻— and Br⁻— dependent oxidation reactions in the asthmatic state, because apoptotic cell death is much less “proinflammatory” than is cell necrotic death. Therefore, HOSCN production is preferred over that of NO₂⁻ and HOBr in the asthmatic state as HOSCN is relatively innocuous to small airway epithelial cells in comparison with NO₂. and HOBr. Moreover, even in the event that these cells are exposed to high concentrations of HOSCN (ca.150 μM based on preliminary experiments), the resulting cell death would be apoptotic and not necrotic, and this would minimize local inflammation.

### III. Effects of SCN⁻ Supplementation on EPO-Mediated Activity

### Example 1

### Attenuation of Br⁻ and NO₂⁻-Based EPO Toxicity for Small Airway Epithelial Cells by SCN⁻

Given that NO₂⁻ and Br⁻ compete with SCN⁻ for oxidation by EPO, experiments were performed to ascertain the effect of increasing SCN⁻ concentrations upon the Br⁻- and NO₂⁻-based toxicity of the EPO system. Two clinically relevant cell lines, BAES-B2 and SAEC (Clonetics) were used. A 2 cm² monolayer of ⁵¹Cr-labeled cells were first exposed to 100 μL of 100 nM EPO for 15 minutes, then unbound EPO was washed away leaving cell surface-coated monolayers. Monolayers were then overlaid with H & H buffer supplemented with either 100 μM NaNO₂⁻ (open boxes), or 100 μM NaBr (closed boxes), and increasing concentrations of SCN⁻. These levels of NaNO₂ or NaBr represent upper limits of levels found in inflammatory fluids. Either 100 μM (for NO₂⁻) or 33 μM (for Br⁻) H₂O₂ was then added. After 2 hours of incubation, ⁵¹Cr release was assayed. Cellular monolayers were solubilized, samples were centrifuged, and gamma counts determined. Results were similar for both BAES-B2 and SAECs. For both NO₂⁻ and Br⁻, in the absence of SCN⁻, chromium release was approximately 50% of the total chromium. The Br⁻ based toxicity of EPO was nearly completely blocked by SCN⁻ concentrations of ≧10 μM; NO₂⁻-based toxicity was severely attenuated by SCN^{- ≧33} μM. Therefore, normal serum concentrations of SCN⁻ (10-100 μM) completely abrogated Br⁻ based toxicity and strongly attenuated (80-85%) NO₂-based toxicity (ID₅₀ of SCN⁻ for Br⁻ and NO₂⁻: 3 μM and 15 μM, respectively). Physiological concentrations of SCN⁻ strongly inhibit or block toxicity otherwise seen in the presence of physiologic concentrations of Br⁻ or NO₂⁻. These results implicate EPO in oxidative protein damage in asthma.

### Example 2

### SCN⁻ Severely attenuated HETE Generation From PAPC Vesicles

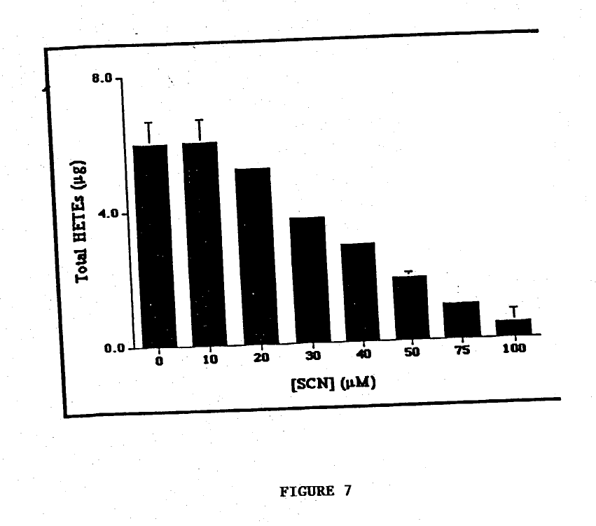

The EPO/NO₂⁻/H₂O₂ system generates reactive nitrogen species that generates bioactive eicosanoids, e.g. hydroxyeicosatetraenoic acids (HETEs) from unilamellar vesicles comprised of 1-palmitoyl-2-arachidonyl-sn-glycero-3-phosphocholine (PAPC). To determine if SCN⁻ affects HETE generation from PAPC vesicles, the following experiment was performed. Reaction mixtures consisting of 0.8 mg lipid/mL PAPC, 60 nM EPO, 100 μM NO₂⁻, 100 ng/mL glucose oxidase, 100 μg/mL glucose were incubated for 2 hours at 37° C. HETEs were analyzed by lipid extraction and HPLC with on-line electrospray ionization tandem mass spectometry (LC/MS/MS). Results in FIG. 7 indicate that physiological concentration of SCN⁻ severely attenuated HETE generation from PAPC vesicles. The EPO/SCN⁻/H₂O₂ system (100 μM SCN⁻) generated no detectable HETEs from PAPC vesicles. These data suggest that to blunt EPO-dependent tissue damage it may be advantageous to increase SCN⁻ levels not just to normal but to high-normal or above.

Therefore, HOSCN imposes oxidative stress primarily by reacting with intracellular glutathione and critical sulfhydryl-modifiable enzymes; NO₂. reacts with lipids to cause lipid peroxidation reactions and breaching of membrane integrity; and HOBr reacts with membrane protein components to destroy membrane integrity. In addition, low serum SCN⁻ levels increase the risk factor for development of Br⁻ and NO₂⁻-based EPO-mediated oxidant damage in the bronchoalveolar epithelium of asthmatics suggesting that dietary augmentation of serum SCN⁻ to normal or supraphysiologic levels can attenuate the generation of these cytotoxic oxidants and improve the clinical severity of asthma.

### Example 3

### Effect of SCN⁻ on Peroxidase-Mediated NO Consumption

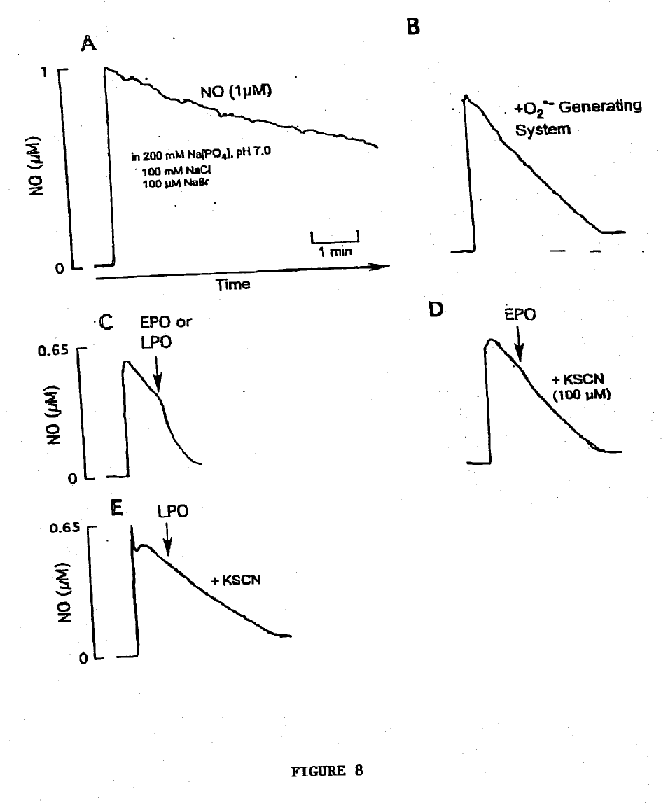

NO decay was determined in phosphate buffer in the absence versus presence of plasma levels of halides using an NO-selective electrode. NO-saturated buffer was added to a continuously stirred reaction mixture. FIG. 8A illustrates NO decay by autoxidation. FIG. 8B illustrates the accelarated NO decay in the presence of an O₂^{.-} generating system (lumazine, Xanthine oxidase). FIG. 8C illustrates the rapid consumption of NO following addition of either EPO or LPO to reaction mixtures prepared as in FIG. 8B. FIGS. 8D and 8E illustrates the SCN⁻ mediated attenuation/inhibition of peroxidase-mediated NO consumption. These results indicate that SCN⁻ salts function in vivo to prevent NO consumption by peroxidases, and hence promote the beneficial action of NO.

MPO, EPO, and LPO all catalytically consume NO, a bronchodilator in the presence of physiological relevant concentrations of Cl⁻, Br⁻ and NO₂⁻ but not in the presence of high physiologic levels of SCN⁻.

### Example 4

### Effect of SCN⁻ on Tracheal Rings Relaxation and Heart Failure

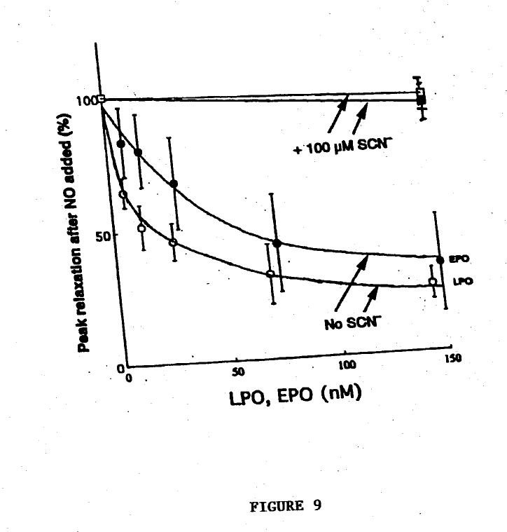

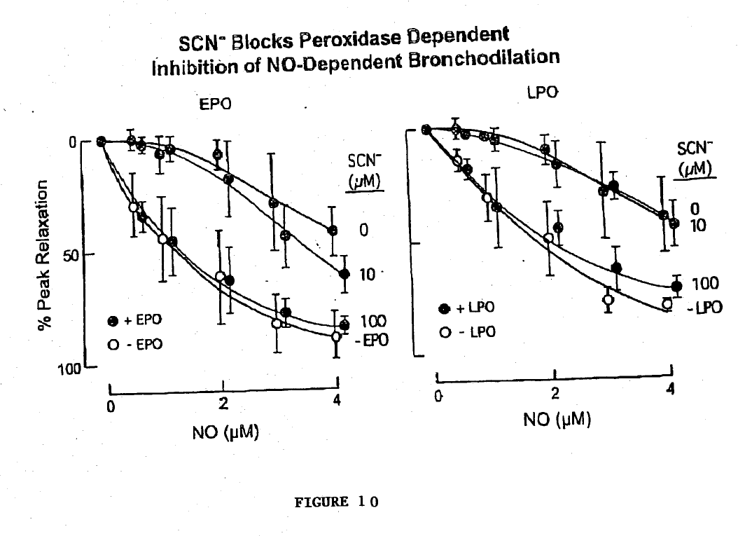

Isolated rat bronchial rings were preconstricted with methacholine, then exposed to NO without 100 μM SCN⁻ (FIG. 9, bottom two curves) or in the presence of 100 μM SCN⁻ (top two curves). Results indicate that preconstricted bronchial tracheal rings do not undergo relaxation in the presence of NO in the presence of LPO, or EPO, but do so when high physiologic concentrations of SCN⁻ are added to compete with NO.

FIG. 10 illustrates the effects of 10 and 100 μM SCN⁻ on peroxidase-treated tracheal rings. The ability of peroxidases to block NO consumption was affected by physiologically relevant levels of SCN⁻. These results suggest that SCN⁻ salts can serve as inhibitors of peroxidase activity in vivo.

In an isolated, profused rat heart model of EPO-mediated heart failure, the presence of ≦10 μM SCN⁻ abrogated acute congestive heart failure that was caused by EPO in the presence of H₂O₂ and 100 μM Br⁻.

### Example 5

### Effect of SCN⁻ Supplementation on Br—Y Levels and Mucus Severity Index of Bronchioles in Murine Allergen Challenge Model of Asthma

Mice were sensitized by intranasal instillation of Aspergillus and aeroallogen challenged one month later with aerosol saline (control) or Aspergillus (antigen challenged). At 2 days before the one-month aeroallogen challenge, one group of antigen-challenged animals received i.p. 0.5 mL of 0.1% KSCN and 0.1% KSCN in drinking water. Three days after the aeroallogen challenge, both groups were challenged with intranasal inoculums of Candida and 6 hours later subjected to BAL and the content of supernatant protein Br—Y determined using stable isotope dilution GC-MS. Br—Y is a biomarker of EPO-dependent generation of HOBr, which is highy injurious to mammlian cells. The EPO-catalyzed product of SCN⁻ oxidation, HOSCN, is not. Histologic sections of lungs obtained at time of lavage were stained for PAS and evaluated for mucus severity index as decribed in J Immunol 167:1672-82, 2001. Mucus is a clinical hallmark of asthma and correlates directly with asthma severity in humans and animals. Results are shown in Table 2.

TABLE 2
Effect of SCN⁻ supplementation on Br—Y levels and mucus severity
index of bromochioles in murine allergen challenge model of asthma

| | Lavage fluid | | |
|---|---|---|---|
| | bromotyrosine | | |
| | (μmol Br—Y/ | Mucus index ± | |
| Experimental group | μmol Y) | S.D. | comments |
| Wild type mouse, | 0 ± 1 | 0.21 ± 0.18 | |
| saline challenge | | | |
| N = 5 | | | |
| Wild type mouse, | 1433 ± 391 | 8.8 ± 1.8 | |
| antigen challenge | p < 0.0001 | p < 0.001 | |
| N = 5 | vs saline) | vs saline | |
| Wild type mouse, | 196 ± 103 | 3.4 ± 1.4 | Mucus index 61% |
| SCN supplemented, | p < 0.0001 | p < 0.001 | reduced from w.t. |
| antigen challenge | vs antigen- | vs. antigen- | antigen- |
| N = 5 | challenged | challenged | challenged; |
| | | | BrY 86% reduced |
| EPO k.o. mouse, | 0 ± 1 | 0.1 ± 0.1 | |
| saline challenge | | | |
| N = 5 | | | |
| EPO k.o. mouse,, | 0 ± 1 | 2.9 ± 1.2 | Mucus index 70% |
| antigen challenge | p < 0.0001 | p < 0.001 | reduced from w.t. |
| N = 5 | vs w.t. | vs. w.t. | antigen- |
| | antigen- | antigen- | challenged; |
| | challenged | challenged | BrY 100% |
| | | | reduced |

These data show: 1) EPO plays a major role in inducing mucus production in this murine model of asthma; and 2) supplementation with SCN⁻ essentially completely eradicates the contribution of EPO to increasing the mucus severity index (i.e., 61% reduced versus 70% reduced in SCN-supplemented wild type versus EPO k.o.). Neutrophil myeloperoxidase (MPO) also contributes to mucus formation because MPO k.o. mice also had an attenuated mucus severity index versus wild type.

### Example 6

### Dietary Supplementation With SCN⁻ Blocks EPO-Mediated Increase in Lung Bromotyrosine Levels in Response to Aeroallergen Challenge in Guinea Pigs

To test the hypothesis that dietary supplementation with SCN⁻ blocks EPO-dependent accumulation of protein Br—Y in an aeroallergen challenge model of asthma, three groups of 6 Hartley guinea pigs were sensitized to ovalbumin (OVA) by i.p. injection of 0.5 ml of 0.1% OVA and 10% aluminum hydroxide adjuvant on day one. A second sensitization with OVA was performed on day 14. One group of 6 animals was given an i.p. injection of 3.125 ml of 0.4% sodium thiocyanate (NaSCN) on day 27 and had its drinking water subsequently supplemented with 0.2% NaSCN (“SCN”; n=6). On day 28, animals were challenged with aerosolized normal saline (“control”; n=6) or aerosolized 0.1% OVA solution (not supplemented with thiocyanate=“OVA”; n=6; and SCN-supplemented=“SCN”; n=6) for one minute on five successive occasions, each separated by a minute of recovery. Two days later, the animals were sacrificed, and their lungs were lavaged with normal saline, excised, and snap-frozen in liquid nitrogen.

Specimens were subsequently analyzed for EPO-specific bromotyrosine (Br—Y) and myeloperoxidase-specific chlorotyrosine (Cl—Y) in acid hydrolysates using stable isotope dilution HPLC/GC mass spec as described above for the mouse experiments. Data are expressed as ratios of bromotyrosine to native tyrosine (Br—Y/Y) or chlorotyrosine to native tyrosine (Cl—Y/Y) to correct for differences in protein concentration.

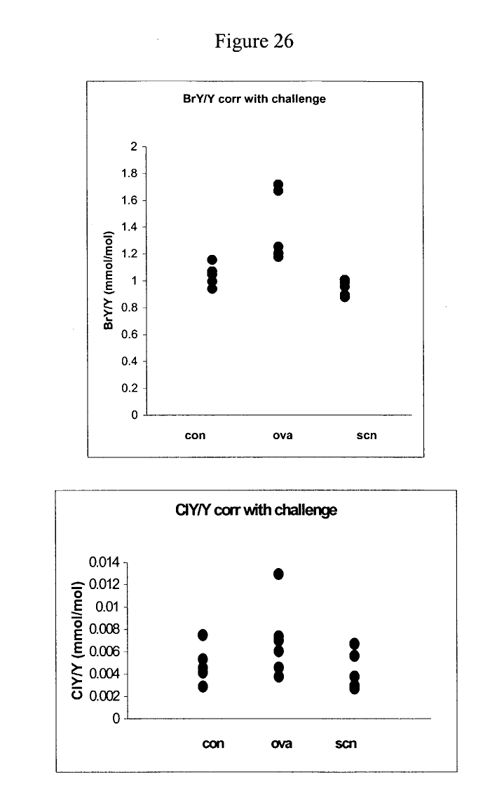

As shown in FIG. 26, top panel, where each animal is represented by a separate dot, control animal lungs (“con”) had mean Br—Y/Y levels of 1.04 mmol/mol Br—Y. This is an extremely high level comparable to that of critically ill, ventilator-dependent human asthmatics and aeroallergen-challenged mice (see above in Table 2). Levels of Br—Y/Y rose to 1.37 mmol/mol after OVA challenge (“ova”, p<0.02 vs. control), which was completely abrogated (mean 0.93 mmol/mol, p<0.02 vs. OVA) by dietary supplementation with SCN⁻ prior to OVA challenge (“scn”). As shown in FIG. 26, bottom panel, a similar pattern was seen in the case of Cl—Y, where control, OVA, and SCN animals had mean Cl—Y/Y ratios of 0.005, 0.007, and 0.004, respectively (SCN p<0.05 vs. OVA). Thus, although guinea pig lungs at baseline have extremely high EPO-dependent Br—Y/Y ratios suggesting ongoing EPO-mediated protein damage, the highly significant increase that otherwise occurs in response to OVA challenge was completely blocked by dietary supplementation with SCN⁻, similar to what was seen in the murine inflammation model (see Table 2 above). Moreover, SCN⁻ supplementation also blocked myeloperoxidase (MPO)-dependent increases in chlorination-dependent oxidant protein damage. These findings in guinea pig, along with the murine model data, suggest that SCN⁻ supplementation can ameliorate both neutrophil MPO-dependent and eosinophil EPO-dependent protein oxidative damage that occurs during the course of inflammation.

### IV. EPO Activity in Asthmatics

### Example 1

### Detection of Br⁻ and NO₂⁻-Based EPO-Dependent Toxic Oxidants in the Bronchoalveolar Lavage Protein of Asthmatics

An assay based on HPLC and GC-mass spectrometry (GC/MS) was devised for detection of peroxidase-derivatized tyrosine residues in biologic tissues. A technique for the detection of chlorotyrosine (Cl—Y), a specific marker for MPO oxidation, described in Hazen et al. (1997) Free Rad Biol Med 23:909-916, was extended to the EPO. Since other peroxidases such as MPO cannot catalyze the formation of Br—Y in any appreciable quantity at physiologically relevant levels of Br⁻ (<100 μM) bromo- and dibromotyrosine (Br—Y) are very specific markers for EPO oxidation. Large amounts of NO₂—Y also can be generated by the EPO/NO₂⁻ system in the presence of protein (see Wu et al. (1999) J Biol Chem 274:25933-25944), although generation of nitrosotyrosine residues is severely inhibited as competing SCN⁻ levels are increased from subphysiologic to high physiologic concentrations (i.e., up to 100 μM). The EPO catalyzed-oxidation of NO₂⁻ likely yields NO₂., a potent initiator of lipid peroxidation. It was shown that this system is capable of generating large amounts of lipid hydroperoxides from model liposomes and intact cells. Indeed, biologically active eicosanoids (i.e., HETEs) and other lipid peroxide derivatives are generated by the EPO/NO₂⁻ sytem. Thus, both Br⁻ and NO₂⁻-based oxidants generated by EPO have the capacity to modify proteins and lipids in a manner that is likely to cause damage and amplify inflammation.

### Example 2

### EPO-Dependent HOBr is Generated in Acute Asthmatic Attacks

Using the technique described above, BAL fluid from critically ill asthmatics admitted to an Intensive Care Unit and intubated was examined for the levels of BR—Y, NO₂—Y and Cl—Y. The control group was a group of ventilated and intubated individuals who had no asthma prior to undergoing elective surgery. Using the GC/MS methodology Cl—Y, a marker of MPO oxidation, and bromo- and nitrotyrosine (NO₂—Y), markers of EPO oxidation, were determined in asthmatics and controls. Results are shown in Table 3.

TABLE 3
Peroxidase-derived oxidant markers in normal and asthmatic bronchial
lavage proteins

| | 3-chlorotyrosine/ | 3-nitrotyrosine/ | 3-bromotyrosine/ |
|---|---|---|---|
| | tyrosine | tyrosine | tyrosine |
| | (μmol/mol) ± | (μmol/mol) ± | (μmol/mol) ± |
| | SEM | SEM | SEM |
| Normals | 65 ± 20 | 52 ± 15 | 13 ± 4.2 |
| (n = 12) | | | |
| Asthmatics | 161 ± 27 | 480 ± 60 | 1090 ± 137 |
| (n = 11) | | | |

Levels of Br—Y, the most specific marker of EPO peroxidation, increased 100-fold, while NO₂—Y and the MPO-specific marker Cl—Y increased 10-fold and 2-fold, respectively, in asthmatics compared to normals. Therefore, EPO-dependent HOBr is generated in acute asthmatic attacks. Protein nitration, whether due to EPO oxidation of NO₂⁻ or NO also occurred. Similar, though less dramatic increases (i.e. 10-fold increases) in Br—Y occurred after subsegmental allergen challenge in human asthma (see Wu et al (1999) J Clin Invest 105:1455-1463). These results demonstrate the importance of EPO-dependent reactions in the pathogenesis of asthma.

### Example 3

### Correlation of EPO Activity With Lung Function in Asthmatics After Allergen Challenge

Br—Y, a specific marker of EPO activity, increases in bronchoalveolar lavage after segmental allergen (Ag) challenge or acute asthma exacerbation. To determine if EPO activity, as measured by serum Br—Y, correlates with airway reactivity in asthma, the following experiment was performed. Whole lung Ag challenge on 8 atopic asthmatics was carried out and serum levels of protein-bound Br—Y before, and 24 and 48 hours after challenge were determined. Results indicate that Br—Y did not change after Ag challenge (all p>0.05), although the forced expiratory volume in 1 s (FEV₁) decreased immediately after Ag in all 8 individuals and 5 of the 8 exhibited a late asthmatic response (FEV₁ at 6 hour was lower than baseline, p<0.05). However, baseline Br—Y inversely correlated with severity of airflow limitation (Br—Y versus FEV₁/FVC ratio at baseline and after Ag, all R<−0.5; all p<0.05). In the 5 individuals with late asthmatic response, baseline Br—Y was inversely correlated with several indices of airflow including baseline % FEV₁ (R=−0.79; p<0.05), % SGaw (R=−0.70; p<0.05), and FEV₁/FVC ratio (R=−0.81; p<0.05). Further, Br—Y in the 5 individuals with late asthmatic response also correlated inversely with indices of airflow at all times after Ag challenge (all R<−0.7, all p<0.05). Therefore, Br—Y correlates with severity of airflow limitation in asthma. High Br—Y may specifically predict those asthmatics with prolonged oxidative stress, i.e., those who develop a late asthmatic response.

### V. Low Levels of Serum SCN in Eosinophilic and Asthmatic Patients

### Example 1

### Serum SCN⁻ Levels in Patients With Hypereosinophilic States

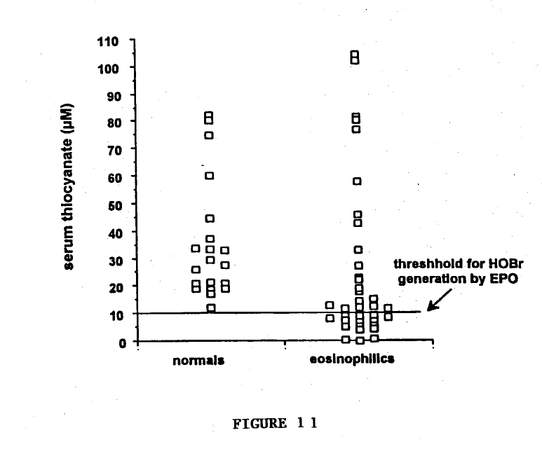

To determine if patients with pronounced (>1500/μL³) eosinophilia have abnormally depressed serum SCN⁻ levels that might put them at risk for generation of HOBr and NO₂. instead of HOSCN, serum SCN⁻ levels in eosinophillic patients and non-eosinophilic patients (normals) were compared (see FIG. 11). The method was that of Boxer and Richards (Boxer et al. (1952) Arch Biochem 39:292-300). The method was based upon (i) quantitative conversion of SCN⁻ to CN⁻ in deproteinized serum in mild oxidation, (ii) trapping of volatile CN⁻ in base solution, and (iii) determination by derivatization of pyridine and malononitrile to a strongly chromogenic substance that can be assayed spectrophotometrically (see Grgurinovich (1982) J Analyt Toxicol 6:53-55). The eosinophilic patients had a variety of clinical conditions, primarily hypereosinophilic syndrome; four had asthma. The mean value for normals was 29 μM and for eosinophilics 12 μM. More importantly, serum SCN⁻ levels of 16/38 (42%) eosinophilics, but no normals (N=20), were severely depressed below 10 μM (p<0.001 by Chi squared analysis). All 4 asthmatics had low serum SCN⁻ levels (4.5, 8.8, 9.2, and 13.3 μM). These results suggest that serum SCN⁻ levels in patients with eosinophilia, including asthmatics can be strikingly depressed. These results also suggest that hypereosinophilic states may, per se, lead to SCN⁻ depletion by EPO-mediated peroxidative depletion because of chronic respiratory burst activation.

### Example 2

### Decreased Levels of Serum SCN⁻ in Asthmatics

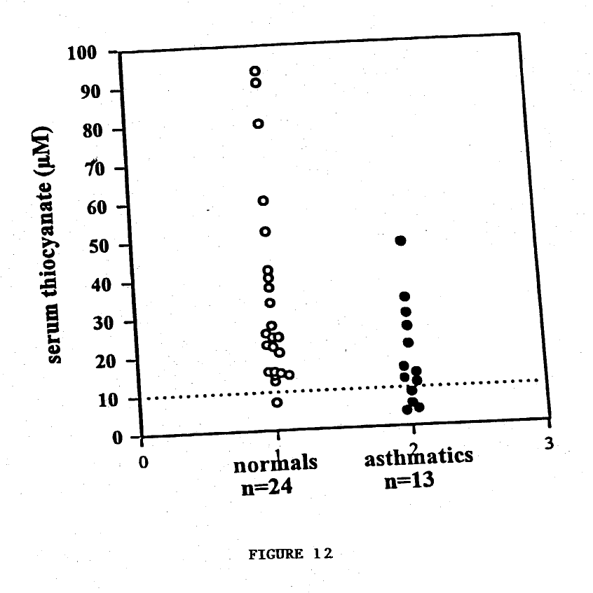

To determine if individuals with eosinophillic states, including asthma, are at risk for depleting serum SCN⁻ levels and therefore entering into a spiral of increasing oxidant damage because of alternative oxidation of NO₂⁻ and Br⁻, an HPLC-based technique for the accurate and sensitive determination of serum SCN⁻ levels was established. Twenty-four normals (non-smokers) and 13 asthmatics, all non-smokers, were selected randomly from the patients of an outpatient asthma clinic. The patients were scheduled for routine (i.e., scheduled, not urgent) follow-up visits. FIG. 12 is a distribution of serum SCNlevels of the 37 patients assessed in the study. Four of the 13 asthmatics but only 1 of the 24 normals had serum SCN⁻ levels below 10 μM, a level at which EPO generation of HOBr becomes prominent. This is significant at a p value of <0.03 by the Fischer Exact Test. Therefore, serum SCN⁻ levels were abnormally low in a significant fraction of asthmatics. This result defines a group of patients potentially at especially high risk for EPO-mediated Br⁻ and NO₂—based toxicity.

### VI. Low Levels of Serum SCN Exacerbate EPO-Dependent Generation of Cytotoxic Oxidants and Asthma Severity

### Example 1

### Guinea Pig Ovalbumin (OVA) Sensitization Model of Asthma

A guinea pig model of asthma is used to study the role of EPO and SCN⁻ in asthma pathology. The sensitization protocol of Santing et al. (see Santing et al. (1994) Clin Exp Allergy 42:1157-63) is used to generate an experimental guinea pig model of asthma that is characterized by a robust, 7-fold increase in BAL supernatant EPO activity over saline control 24h after challenge. This indicates substantial activation and degranulation of bronchial EOs in this preparation. The sensitization protocol involves (i) i.p. injection of 0.5 mL 100 μg/mL OVA and 100 mg/mL Al(OH)₃ and (ii) injecting another 0.5 mL of same s.c. divided over 7 sites in proximity of the lymph nodes in the paws, lumbar area, and neck. Animals are maintained in a specific pathogen-free environment. Challenge is performed 4-6 weeks later by administration of increasing doses of aerosolized OVA aerosol in unanaesthetized animals. During the challenge, lung function is monitored by Buxco (Penh) noninvasively. Challenge is initiated at very low dose of OVA and double doses are used during subsequent challenges. When Penh has doubled, challenge is stopped. In this way, severe reactions possibly leading to anaphylaxis are avoided, obviating the need for antihistamines during the challenge (which may alter subsequent responses). At subsequent time points, invasive mechanics, methacholine or histamine challenge, BAL, phlebotomy, and sacrifice are performed.

### Example 2

### Pulmonary Function Studies

For invasive mechanics measurements, animals (male Hartley guinea pigs, 400-600 gm) are anesthetized with 50 mg/kg ketamine supplemented with 5 mg/kg xylazine. The tracheal intubation is performed through median tracheotomy using thin walled stainless steel tube whose outer diameter matches the internal diameter of the trachea. The tracheal tube has a small side port that is connected to a pressure transducer to monitor airway pressure. Animals' lungs are ventilated with a small animal ventilator set to 6 mL/kg tidal volume and f=40 min⁻¹. After 15-20 minutes to allow stabilization, 0.1 mg of vecuronium is injected in a neck vein to paralyze the animal, preventing corruption of the breathing pattern from spontaneous respiratory movements. Respiratory system resistance (Rrs) is measured using standard techniques as described by Suman et al. (see Suman et al. (in press) J Appl Physiol) and Ray et al. (see Ray et al. (1989) J Appl Physiol 66:1108-1112) after placing the animal in a small animal plethysmograph. Measurements are made every 2 minutes for a minimum of 10 minutes to establish baseline Rrs. A series of 12 breaths are recorded followed by a deep sigh administered using the ventilator to prevent atelectasis. Rrs is the average of the 12 breaths prior to the sigh at each time point. Methacholine challenge is then performed by administering increasing concentrations of methacholine delivered by ultrasonic nebulizer into the inspired gas line of the ventilator. Each dose is given over 30 sec period. The first dose is saline vehicle, delivered by 30 sec of mist inhalation. Rrs is then measured every 2 minutes up to 10 minutes. The value at 6 minutes is used to plot dose-response curves. Following saline, 0.06 mg/mL methacholine is administered (30 seconds), and subsequent doses are increased by doubling concentrations. Concentration is increased until Rrs 6 minutes after dose administration at least doubles compared to the baseline value. Parameters derived from the challenge include the PC₅₀ (concentration at which Rrs increases by 50%, determined by interpolation), and overall slope of the dose-response curve determined from (R_{rs,final}−R_{rs,initial})/R_{rs,initial}/[MC]_{final}.

### Example 3

### BAL and Processing of Whole Lung Specimens

After the last dose of methacholine, lung lavage, dissection and fixation are performed as follows. Blood is first drawn from venous catheter previously inserted in a neck vein. Then 80 mg of pentobarbital is administered i.v, to sacrifice the animal. The chest wall of the animal is opened widely via midline stemotomy. The main stem bronchus to the right lung is occluded using a tight ligature to prevent lavage fluid from entering the right lung. The left lung is then lavaged using a 5 mL syringe filled with warmed saline. The syringe is pumped 5 times and fluid saved from the last withdrawal and immediately iced. The lungs are then perfused via the pulmonary artery using warmed saline, with effluent draining from the opened left atrium. When effluent appears clear, perfusion is stopped. The left lung is fixed in situ by instilling formaldehyde into the trachea. The right lung is then snap-frozen by first cutting it free on the proximal side of the ligature (to insure continued inflation during processing), then immersion into liquid nitrogen.

BAL fluid is analyzed for cell count and differential, pelleted, and the supernatant fluid divided in two. Specimens are snap frozen on liquid nitrogen. The pellet and one supernatant aliquot are assayed for Cl—Y, Br—Y, and NO₂—Y. The remaining supernatant fluid aliquot are assayed for Br⁻, NO₂⁻, and SCN⁻, as well as urea for calculation of epithelial lining fluid volume, EPO activity, lipid peroxidation as 8-isoprostanes, and carbonyl carbon formation (see below). The unfixed, snap-frozen lung is assayed for Cl—Y, Br—Y, and NO₂—Y. The fixed lung is assessed histologically for asthma severity. Serum is assayed for Br⁻, NO₂⁻, and SCN⁻, as well as urea for calculation of epithelial lining fluid volume.

### Example 4

### Assay of SCN⁻ in Serum and BAL Fluid

As described by Michigami et al. (1988) Analyst 113:389-392, serum specimens are diluted with an equal volume of distilled water, supplemented with the addition of an equal volume of acetonitrile to precipitate proteins and solubilize SCN⁻. Specimens are centrifuged and transferred after filtering directly into an HPLC column using TSK-gel IC anion exchange (50×4.6 mm inner diameter) column using potassium phosphate buffer pH 6.5 as an eluent with a flow rate of 1 mL/minute. In this system SCN⁻ has a retention time of 11 minutes when detected at 195 nm with UV detection apparatus. This assay was found to have a sensitivity of down to 5 ng/mL (approximately 15 nM) and at 100 ng/mL (approximately 1.5 μM) the standard deviation for determinations was 1.50%. Given that normal serum levels using the colorimetric assay are between 10-100 μM this HPLC technique can detect serum SCN⁻ levels at 1/100^{th} the lower limit of normal. For BAL specimens, on the day of the lavage, guinea pigs are subjected to a blood urea nitrogen and albumin determination. Urea nitrogen and albumin are also quantitated in the lavage fluid return to allow accurate assessment of the volume of epithelial lining fluid. Even after the 20-50 fold dilution that occurs with the lavage fluid, the HPLC assay still allows for submicromolar sensitivities, more than adequate for these purposes. Should it prove necessary, even more HPLC sensitive assays based on fluorescence derivatization are available.

### Example 5

### Method for Detection of NO₂⁻ and Br⁻ in Serum

An HPLC technique for the simultaneous determination of NO₂⁻ and Br⁻ in human serum by ion chromatography using an ODS (octadecyl silica) column dynamically coated with cetyl pyridinium chloride and UV detection at 210 nm described by Michigami et al. (see Michigami et al. (1989) Analyst 114:1201-1205) is used. The eluent is 1 mmol citrate solution in 2.5 methanol at pH 6.5. The sensitivity of this assay is at the submicromolar level. The Griess reagent (see Feder et al. (1997) Am J Respir Cell Mol Biol 17:436-442) for measuring NO₂⁻ levels in serum and other biologic specimens is also used.

### Example 6

### Assay of EPO Activity, Lipid Peroxidation, and Carbonyl Carbon, and β₂-Macroglobulin in Bronchoalveolar Lavage and Whole Lung Homogenates

To characterize the time-course of EO and EPO accumulation in the BAL fluid, peroxidase activity and protein levels are assessed. Peroxidase activity is assayed using the method of Tagari et al. (see Targari et al. (1993) J Immunol Methods 163:49-58) that involves the oxidation of 3,3′5,5′-tetramethylbenzidine (TMB) in the presence or absence of 3 mM NaBr. Br⁻ mediated TMB oxidation is selectively stimulated by EPO, i.e., MPO of neutrophils is inactive under conditions of the assay, therefore total lung neutrophil MPO and EPO activity can be distinguished.

CM-52 resin chromatography and a NaCl gradient (see Thomas et al. (1994) J Dent Res 73:544-555) also are used to distinguish and separate different peroxidases. In initial experiments guinea pig BAL specimens are passed through a CM-52 resin to separates MPO from EPO from LPO. The contribution, if any, of LPO to total peroxidase activity also is determined. A sandwich ELISA utilizing polyclonal affinity-purified rabbit antibodies also is used for the detection/quantitative-assessment of guinea pig EPO. Assay of lipid peroxidation as 8-ispoprostanes and protein carbonyl carbon formation is performed as described. BAL fluid α₂-macroglobulin, a measure of tissue damage and plasma extravasation, is determined using immunoassay as described by Greiff et al. (see Greiff et al. (1998) Thorax 1010-1013).

### Example 7

### Determination of MPO- and EPO-Specific Endproducts in BAL Pellet Supernatant Fluid, and Snap-Frozen Whole Lung

Cl—Y, Br—Y and NO₂—Y residues in whole lung homogenates and BAL fluid are assessed using the isotope dilution mass spectrometry methodology as described in Hazen et al. (1997) Free Rad Biol Med 23:909-916; Wu et al. (1999) Biochemistry 38:3538-3548; and Funderburk et al. (1967) Am J Physiol 213:1371-1377. Acid hydrolysates are prepared by first precipitating and desalting them in a single-phase extraction mixture composed of H₂O/methanol/H₂O-saturated diethyl ether (1:3:7, v/v/v) as described in Wu et al. (1999) J Biol Chem 274:25933-25944. Prior to initiating hydrolysis, acid mixtures are degassed under vacuum and sealed under blanket of argon. The hydrolysis is performed in 4 N methane sulfonic acid (0.5 mL) supplemented with 1% phenol for 24 hours at 100° C. to avoid formation of trace levels of halogenated tyrosine analogues during acid hydrolysis. GC/MS analyses of HPLC-isolated L-tyrosine and its oxidation products are performed following derivatization to their n-propyl per-heptafluorylbutyryl or n-propyl per-pentafluorylproprionyl derivatives as described in Crowley et al. (1998) Anal Biochem 259:127-135. Negative ion chemical ionization GC/MS studies are performed utilizing a Perkin Elmer (Norwalk, Conn.) TurboMass spectrometer equipped with chemical ionization probe. This assay is sensitive to the 1 fmole range. NO₂—Y also is quantitated by similar methodology (see Wu et al. (1999) J Biol Chem 274:25933-25944; and Rasmussen et al. (1999) Methods in Enzymol 300:124144). Results are expressed as mmol/mol tyrosine as well as mmol/mol tyrosine/unit EPO. The following exemplifies the detection of Br—Y in BAL following OVA challenged in guinea pigs.

Guinea pigs sensitized to OVA were challenged with OVA as described above and their lungs lavaged 24 hours later. Proteins in BAL were then analyzed for the presence of Br—Y by GC/MS. A major peak was noted in the chromatogram when monitoring at the m/z of the base ion for derivatized Br—Y, which has an identical retention time as that observed with labeled synthetic standard. Full-scan negative ion mass spectrum of derivatized 3-Br—Y generated in guinea pig lung following OVA challenge showed characteristic isotopic clusters, and fragmentation pattern that unambiguosly identify the analyte as 3-Br—Y.

The level of HOSCN production in tissue is determined by assaying, for example, derivatization of cysteinyl residues. A mixture of N-acetylated amino acids is exposed to HOSCN/cyanate generated by the EPO/SCN⁻/H₂0₂ system. The resulting products are separated by HPLC after exposure to acid hydrolysis to detect stable end products.

### Example 8

### Histologic Assessment of Asthma Severity in Fixed Lungs

The severity of pulmonary inflammation is assessed by a combination of light and immunofluorescence microscopy for localization of EO granule proteins and for Br—Y and NO₂—Y. Coronal microscopic sections from the formalin-fixed lung (containing central airways) are stained with H & E and serial sections processed for immunofluorescence staining with affinity purified antibody to EO granule major basic protein (MBP) as described in Filley et al. (1981) J Immunol Methods 47:227-238; and Filley et al. (1982) Lancet 2:11-16. Affinity-purified antibody to MBP is ideal for the immunofluorescence staining and for determining the association between MBP deposition and histological damage (see Peters et al. (1983) J Invest Derm 81:39-43). Histological changes are assessed blindly on random sections as described by Underwood et al (see Underwood et al (1995) Eur Respir J 8:2104-2113) utilizing a five point histopathology grade and scoring separately 1) perivascular and bronchiolar eosinophilia; 2) edema; 3) mucin; and 4) epithelial damage. Furthermore, to determine the relationship between EPO deposition and histological changes, rabbit anti-EPO is produced and affinity-purified for EPO localization. Mucin containing cells are quantified after staining with diastase-periodic acid-Schiff as described by Cohn et al. (see Cohn et al. (1997) J Exp Med 186:1737-1747). Antibody to Br—Y is used for immunolocalization of brominated proteins for localization of MBP and EPO. Immunopurified rabbit polyclonal antibodies to nitro-tyrosine are purchased from Upstate Biotechnology, Inc., Lake Placid, N.Y. and used for immunolocalization of nitrated proteins. These methods are used to ascertain whether, in serial sections, areas with high concentrations of EPO-derived oxidative protein damage correspond to areas of histologic damage.

### Example 9

### Production of Affinity-Purified Antibody to Guinea Pig MBP and EPO

For studies utilizing rabbit anti-guinea pig MBP polyclonal antibody, antibody purified by affinity chromatography over a column of MBP is used. Guinea pig EOs are stimulated by repetitive peritoneal lavage as described in Gleich et al. (1973) J Lab Clin Med 82:522-528; and Lindor et al.(1981) J Immunol Methods 41:125-134. Guinea pig MBP is purified from peritoneal EOs as described previously (see Gleich et al. (1973) J Exp Med 137:1459-1471; Gleich et al. (1974) J Exp Med 140:313-332).

EPO is a byproduct of the MBP production from guinea pig EO granules and is purified by chromatography over carboxymethyl Sepharose. Purified anti-EPO is produced by affinity chromatography over a column of EPO in a similar manner to that described for MBP.

### Example 10

### Determination of the Time-Course of BAL and Lung Tissue EPO-Mediated Oxidative Protein Modification in the Guinea Pig Ovalbumin Sensitization Model of Asthma and the Relationship of EPO-Mediated Oxidative Protein Modification to Airways Hyperreactivity and Histologic Asthma Severity

To identify the time of maximum EPO-specific of protein modification after OVA rechallenge (and therefore damage) and its potential relationship to functional and histologic asthma severity, the following experiment is performed.

Guinea pigs are subjected to ovalbumin sensitization using the protocol described above. Six groups of 15 animals (i.e., a total of 90) are assayed at 0, 12, 24, 48, 72, and 96 hours after aerosol OVA challenge. Groups of 6 additional animals (the minimum number required for non-parametric analysis) for each time point (i.e., 36) receive saline instead of OVA challenge and serve as controls. Numbers of animals in each group are determined from power calculations assuming un-paired design. Fifteen animals in each group is sufficient for detecting a change in Br—Y, PC₅₀, and histologic severity index amongst the OVA-challenged animal groups at various time intervals of ≧125% of the S.D. of the experimental groups with 90% power, P<0.05. After challenge with aerosol OVA, animals are assessed for pulmonary function and BAL, lung, and serum specimens obtained and thoroughly analyzed for tyrosine residue endproducts, lipid peroxidation, halide composition, and histologic grading of severity, and BAL β₂-macroglobulin.

The primary purpose of these experiments is to elucidate the time-course of BAL and whole lung EPO activity and EPO-mediated oxidative protein modification after OVA challenge, as assayed by Br—Y levels. A secondary purpose is to relate these EPO-dependent parameters to severity of airways hyperreactivity (as assayed by PC₅₀ of methacholine) and histologic severity of asthmatic changes (as assessed by a histologic scoring system). ANOVA analysis is performed on three primary variables: (i) Br—Y in BAL and whole lung homogenates, (ii) PC₅₀ methacholine, and (iii) asthma histologic severity index across different time points. Time is used as a classification variable to determine if there is an effect of time after OVA challenge on the individual parameters. The time of maximal effect is determined by comparing the time points using an appropriate statistical method (i.e., parametric or non-parametric) based upon ANOVA. Studies are performed at or near the maximum Br—Y level to maximize the sensitivity of discerning differences in Br—Y caused by manipulation of bronchiolar halide/SCN⁻ concentrations, either by intranasal instillation or, alternatively, by dietary manipulation of serum levels. A single “compromise” time point is used to assess Br—Y level and histologic severity maxima if Br—Y and histologic severity maxima are at relatively close time point. In not, separate groups of animals are assessed at the Br—Y maximum and histologic severity maximum.

To establish relationships between EPO-mediated oxidant damage and clinically relevant secondary endpoints, Br—Y levels are correlated with PC₅₀ and histologic severity. This correlation is revealed using linear regression analysis with, for example, PC₅₀ methacholine or histologic lung damage score as the dependent variable and Br—Y concentrations as the independent variable at each time point. The time of maximal correlation is used to identify physiologic relationships, i.e. oxidant (Br—Y) damage precedes and correlates with the magnitude of histologic damage.

Epithelial lining fluid levels of the three relevant potential substrates—Br⁻, NO₂⁻, and SCN⁻ and their relationships to concomitant serum levels are determined. Sputum obtained bronchoscopically showed SCN⁻ levels (0-60 μM) similar to the range in serum. Since Br⁻, like SCN⁻, is freely distributed in extracellular fluid, serum Br⁻ levels determine BAL levels. The histologic localization of Br—Y and NO₂—Y as well as the time course of lipid peroxidation and its relationship, if any, to Br—Y, NO₂—Y, and asthma pulmonary pathology are examined. Therefore, the relationships among EPO-dependent cytotoxic oxidant production, time after antigen challenge, airways hyperreactivity, and histologic severity of asthma, are determined.

### Example 11

### Effect of Intranasal Instillation of Br⁻, SCN⁻, and NO₂⁻ Upon BAL and Lung Tissue EPO-Mediated Oxidative Protein Modification and Pulmonary Pathology in the Ovalbumin Sensitized Guinea Pig Model

The following experiment is performed to determine if by directly manipulating the relative substrate halide composition of the intrabronchial luminal contents, the severity of the resulting EPO-mediated oxidative damage and so the severity of the induced pathologies can be influenced. Solutions containing 1 mM SCN⁻, 1 mM Br⁻ or 100 μM NO₂⁻ (all 10× the upper range of physiologic serum concentrations) are administered intranasally and the severity of the asthmatic response and generation of cytotoxic oxidants are assessed. Measurements of EPO-mediated oxidative damage are taken at a time-point determined from Section VI, Example 10. From the day before initiation of aerosol ovalbumin challenge, four groups of guinea pigs (n=15) are subjected to intranasal instillation of 300 μL of PBS (100 mM Cl⁻), PBS+1 mM NaSCN, PBS+1 mM NaBr, or PBS+100 μmol NaNO₂ in each nostril. Intranasal instillation is performed every 6 hours until the time of analysis. The numbers of animals in each group is determined from power calculations assuming unpaired design. A group size of 15 animals per group allows for detecting a change in Br—Y, PC₅₀, and histologic severity index amongst the OVA-challenged animal of ≧125% of the S.D. of the experimental groups with 90% power, P<0.05.

As for Section VI, Example 10, pulmonary functions, and chloro-, bromo-, and NO₂—Y in BAL and whole lung homogenates are determined. Lung histology is assessed and serum and BAL specimens are assayed for levels of Br⁻, NO₂⁻, and SCN⁻. SCN⁻ ameliorates, while Br⁻ and NO₂⁻ increase, the levels of Br—Y and NO₂—Y as well as the severity of the resulting asthma. There is an inverse correlation between (i) SCN⁻ levels and (ii) Br—Y, NO₂—Y levels, and asthma severity. ANOVA followed by selective t-tests are used to test for differences in PC₅₀ methacholine, histologic severity, and Br—Y and NO₂—Y levels in BAL and whole lung among the four treatment groups. Multiple linear regression is used to determine factors responsible for changes in PC₅₀ or histologic severity of airway damage. PC₅₀ or histologic severity is treated as a continuous variable and correlated Br—Y and NO₂—Y levels in BAL and whole lung by multiple regression.

### Example 12

### Establishment of Protocols for Selective Depletion and Augmentation of Serum SCN⁻ Levels in Guinea Pigs

A protocol to manipulate SCN⁻ levels from just prior to aerosol challenge with OVA through the period of ultimate analysis of Br—Y and asthma severity is developed. The protocol assures that alteration of SCN⁻ levels affect only the inflammatory phase after OVA re-challenge and not earlier immune responses to the primary immunization with OVA.

Guinea pigs have serum SCN⁻ levels similar to that of humans (i.e., 20-100 μM). To establish an experimental protocol that selectively depletes SCN⁻ over a period of roughly six days, levels of SCN⁻ in rodents are depleted by dietary supplementation with potassium perchlorate as described in Funderburk et al. (1967) Am J Physiol 213:1371-1377. Br⁻, NO₂⁻, and SCN⁻ levels are measured after perchlorate administration. Dietary supplementation with either NO₂⁻ or Br⁻ is added to the perchlorate depletion regimen if dietary supplementation with perchlorate alone reduces NO₂⁻ and Br⁻ as well as SCN⁻ levels.

I.p. injection of 5 mg/kg of KClO₄ with subsequent supplementation of drinking water with 1% perchlorate leads to rapid urinary excretion of SCN⁻ and reduction of serum levels to <2 μM for a two-week period. Serum SCN⁻, NO₂⁻ and Br⁻ levels in guinea pigs (n=10 animals/group) are measured. A 500 μL specimen is obtained from the vena cava of anaesthetized animals by phlebotomy. Subsequently animals are injected i.p. with perchlorate and fed drinking water supplemented with perchlorate as described above. Additional serum specimens are obtained from a given group of guinea pigs either on days 1, 3, 5, and 7 days thereafter for assay of these same anions.

Depletion of Br⁻ and NO₂⁻ are abrogated by supplementation with NO₂⁻ or Br⁻. Animals are perchlorate treated, then subsequently supplemented with either NO₂⁻ or Br⁻ in the drinking water such that SCN⁻ alone is depleted without concomitantly affecting Br⁻ or NO₂⁻. To show that any effects observed are due specifically to depletion of SCN⁻, animals are first depleted of SCN⁻, then dietary supplementation with SCN⁻ is given to restore normal levels (30-100 μM). To selectively elevate serum SCN⁻ to supraphysiologic (ca. 300 μM) levels, animals are given drinking water supplemented with 1% NaSCN. Serum SCN⁻, NO₂⁻ and Br⁻ levels are then determined.

### Example 13

### Effect of SCN⁻ Depletion and Supplementation Upon BAL and Lung Tissue EPO-Mediated Oxidative Protein Modification and Pulmonary Pathology in the Ovalbumin Sensitized Guinea Pig Model

Four experimental groups, each comprised of 15 guinea pigs are utilized for these studies. The number of animals in each group was determined from power calculations assuming un-paired design. A group size of 15 animals allows for detecting a change in Br—Y, PC₅₀, and histologic severity index amongst the OVA-challenged animal groups of ≧125% of the S.D. of the experimental groups with 90% power, P<0.05.

The first group is the control and receives KCl in lieu of KClO₄ to control for the effect of potassium. The second group is the SCN⁻ depletion group and is subjected to KClO₄ treatment alone or perchlorate plus appropriate Br⁻ or NO₂-supplementation to maintain physiologic levels. The third group of animals is SCN⁻ depleted (via perchlorate) as in group 2, with supplementation of SCN⁻ to achieve normal (30-100 μM) levels to demonstrate that the induced pulmonary pathology occurring in group 2 is due solely to the absence of SCN⁻. The fourth group is supplemented with SCN⁻ to maintain levels at supraphysiologic (i.e., 200-300 μmol) levels. Perchlorate treatments are initiated three days prior to OVA aerosol challenges and maintained throughout the experimental period until the time of analysis. Specimens are analyzed according to the protocol described. The experimental groups are compared using an appropriate statistical method (i.e., parametric or non-parametric) based upon ANOVA. Abnormally low levels of serum SCN⁻ exacerbate, while elevated levels ameliorate BAL and lung tissue EPO-mediated protein modification and pulmonary pathology.

All four groups are then pooled to determine correlations between (i) methacholine challenge PC₅₀ and (ii) BAL and whole lung Br—Y and NO₂—Y levels. Correlations were also determined between (i) histologic severity and (ii) BAL and/or whole lung Br—Y and NO₂—Y levels. In addition, correlations between (i) serum and BAL Br⁻, NO₂⁻, and SCN⁻ levels and (ii) BAL Br—Y and NO₂—Y levels are determined using appropriate multiple regression analysis. Correlations between (i) serum and BAL Br⁻, NO₂⁻, and/or SCN⁻ levels and (ii) whole lung Br—Y and NO₂—Y levels are determined using appropriate multiple regression analysis. Univariate analysis is used to correlate serum and BAL SCN⁻, Br⁻, and NO₂⁻ levels to determine if serum levels determine BAL levels of Br⁻ and SCN⁻. Because NO₂⁻ can be generated in situ in bronchi by iNOS, there may be a poor correlation between serum and BAL levels of NO₂⁻. SCN⁻ will reduce Br—Y and NO₂—Y levels as well as functional and histologic pulmonary pathology. There is an inverse correlation between (I) BAL and serum SCN⁻ levels and (ii) Br—Y and NO₂—Y levels and asthma severity.

Therefore, the effect of manipulating EPO substrates upon the generation of Br—Y and NO₂—Y in the OVA sensitization model of asthma is examined. The time course EPO-mediated protein modification in asthma is determined as well as its temporal and correlative relationship to pulmonary pathology. In addition, Br⁻ and SCN⁻ levels in bronchial fluid and their relationships to serum levels are assessed. Furthermore, the feasibility of dietary intervention as a strategy for manipulating the bronchiolar milieu is determined.

### VII. Nitric Oxide is a Physiological Substrate for Mammalian Peroxidases

### Example 1

### Materials

NO gas was purchased from Matheson Gas products, Inc., and used without further purification. For each experiment, a fresh saturated stock of NO was prepared under anaerobic conditions. The extent of nitrite/nitrate (NO₂⁻/NO₃⁻) build-up in NO preparations over the time course used for the present studies was <1-1.5% (per mol NO), as determined by anion exchange HPLC under anaerobic conditions (Thayer & Huffaker (1980) Anal Biochem 102:110-119). All other reagents and materials were of the highest purity grades available and obtained from Sigma Chemical Co (St. Louis, Mo.) or the indicated source.

Human EPO was isolated from porcine whole blood obtained fresh at the slaughterhouse as described by Jorg et al. (1982) Biochim Biophys Acta 701:185-191. EPO was assayed by guaiacol oxidation (see Klebanoff et al. (1984) Methods Enzymol. 105:399-403). Purity of EPO preparations was assured before use by demonstrating a RZ of >0.9 (A₄₁₅/A₂₈₀), SDS PAGE analysis with Coomassie Blue staining, and in-gel tetramethylbenzidine peroxidase staining to confirm no contaminating MPO activity (van Dalen et al. (1997) Biochem J 327:487-492). MPO was initially purified from detergent extracts of human leukocytes by sequential lectin affinity and gel filtration chromatography as described (Rakita et al. (1990) Biochemistry 29:1075-1080. Trace levels of contaminating EPO were then removed by passage over a sulphopropyl Sephadex column (Wever et al. (1981) FEBS Lett 123:327-331). Purity of isolated MPO was established by demonstrating a RZ of 0.87 (A₄₃₀/A₂₈₀), SDS PAGE analysis with Coomassie Blue staining, and in-gel TMB peroxidase staining to confirm no contaminating EPO activity. Enzyme concentrations were determined spectrophotometrically utilizing extinction coefficients of 89,000 and 112,000 MS⁻¹ cm⁻¹/heme of MPO (Agner (1963) Acta Chem Scand 17:S332-S338) and EPO (Bolscher et al. (1984) Biochimica et Biophysica Acta 784:177-186 and Carlson et al. (1985) J Immunol 134:1875-1879), respectively. The concentration of the MPO dimer was calculated as half the indicated concentration of heme-like chromophore. Bovine LPO was obtained from Worthington Biochemistry Corporation (Lakewood, N.J.) and used without further purification. Purity was confirmed by SDS PAGE analysis with Coomassie Blue staining and also by demonstrating a RZ of 0.75 (A₄₁₂/A₂₈₀).

### Example 2

### NO is Catalytically Consumed by Mammalian Peroxidases Under Physiological Conditions

Initial experiments utilized an NO-selective electrode (ISO-NO Mark II, World Precision Instruments, Sarasota, Fla.) to determine whether NO serves as a general substrate for peroxidases. Reactions were performed under conditions where peroxidases were present in catalytic amounts Experiments were performed at 25° C. by immersing the electrode in 10 mL of 0.2 M sodium phosphate buffer, pH 7.0, under air. NO was added to continuously stirred buffer solution from an NO-saturated stock and the rise and fall in NO concentration was continuously monitored using a chart recorder. To determine the effect of H₂0₂ and peroxidases on NO levels during steady state catalysis, 10 μl H₂0₂ (100 μM final) and 50 μL enzyme (150 nM final) were added to the reaction mixture. Where indicated, solutions were supplemented with NaCl (100 mM) and/or a cell-free O₂^{.-}-generating system comprised of lumazine (0.4 mm) and bovine milk xanthine oxidase (XO, Boehringer Mannheim). Superoxide generation under the conditions utilized was ˜40 μM/minute, as measured by the superoxide dismutase-inhibitable reduction of ferricytochrome c (Lampert and Weiss (1983) Blood 62:645651).

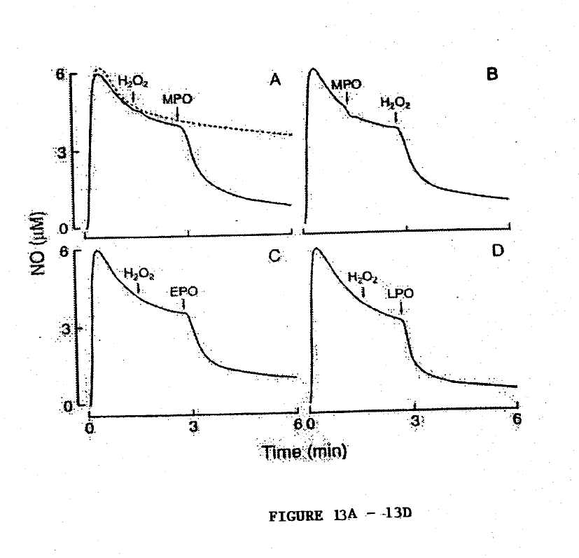

Following addition of an aliquot of NO-saturated buffer to the continuously stirred reaction mixture (6 μM NO final), the NO signal rose rapidly, achieved a maximum after ˜30 seconds, and fell gradually to the origin as NO was depleted by autoxidation (FIG. 13A dotted line). Addition of H₂0₂ to the reaction mixture had no significant effect on the rate of NO decay (FIG. 13A). Subsequent addition of MPO, EPO or LPO to the reaction mixture caused a rapid decay in the level of free NO (FIGS. 13B, 13C, and 13D), indicating that NO is consumed as a substrate by mammalian peroxidases during steady-state catalysis. Reversal of the order of peroxidase and H₂0₂ addition demonstrated a modest brief decrement in NO concentration following addition of only the peroxidase (presumably due to heme Fe(III)-NO complex formation) and then a similar significant acceleration in NO consumption upon subsequent addition of H₂0₂ (FIG. 13B). The concentrations of additions were as follows: H₂O_{2, 100} μM; MPO, EPO or LPO 150 nM. Tracings shown are from a typical experiment performed at least three times.

To examine the potential physiological significance of these observations, the effect of additional substrates on peroxidase-catalyzed consumption of NO was examined. MPO was initially used as a model peroxidase because of its abundance at sites of leukocyte recruitment and activation during inflammation and its well-known use of the abundant halide Cl⁻ as substrate. The rates of NO consumption mediated by MPO in the presence versus absence of plasma levels of Cl⁻ were virtually indistinguishable. These results are consistent with the fact that MPO is far from saturated at plasma levels of Cl⁻. NO consumption by MPO was prevented by pre-incubation of the enzyme solution with sodium azide, a peroxidase inhibitor. Similar results were observed with other mammalian peroxidases.

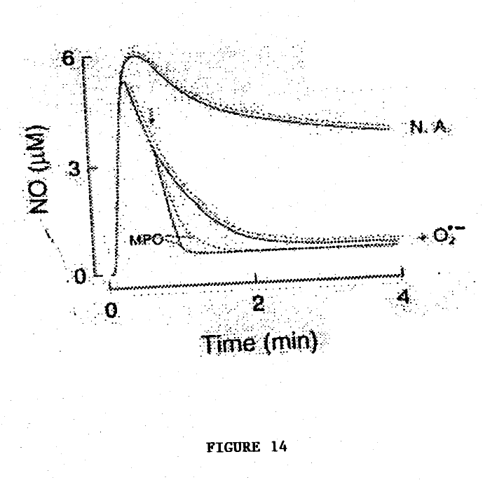

Leukocyte activation in vivo is accompanied by MPO secretion and O₂⁻ formation during the respiratory burst. Since O₂^{.-} interacts rapidly with NO, to determine that peroxidases could accelerate NO consumption during leukocyte activation, the following experiment was performed. Addition of NO to buffer containing a cell-free O₂^{.-}-generating system resulted in accelerated removal of NO, as detected by continuous monitoring with a NO-selective electrode (FIG. 14). Subsequent addition of catalytic amounts of a mammalian peroxidase (data for MPO shown) resulted in the enhanced removal of NO from the buffer (FIG. 14). Similar results were observed with other mammalian peroxidases (data not shown). Finally, addition of MPO to media containing plasma levels of Cl⁻ (100 mM), NO and a O₂^{.-}-generating system resulted in a marked acceleration in the rate of NO consumption above and beyond that observed with that only the O₂^{.-}-generating system (FIG. 14). Thus, peroxidases like MPO can effectively act as catalysts for NO consumption under conditions likely to be physiological in the phagolysosome or at sites of inflammation.

### Example 3

### Spectroscopic and Rapid-Kinetics Characterization of the Interaction Between NO and MPO Compounds I and II

As previously reported, addition of H₂0₂ to MPO-Fe(III) in the absence of co-substrates leads to the accumulation of Compound II via rapid initial formation of Compound I, and subsequent spontaneous one e⁻ heme reduction, (Kettle & Winterbourn (1997) Redox Report 3:3-15) and (Marquez et al. (1990) J Biol Chem 265:5666-5670). Compound II, the rate-limiting intermediate in the peroxidase cycle, possesses a characteristic Soret absorbance peak at 455 nm that is easily distinguished from the Soret absorbance peaks of MPO-Fe(III) and the MPO-Fe(III)-NO. MPO compound II is unstable and converted gradually to the ground state, MPO-Fe(III), within minutes of initiating the reaction.

To examine the kinetics of interaction between NO and MPO Compounds I and II, stopped-flow spectroscopy was used. The kinetics of Compound II formation and decay in the absence and presence of different NO concentrations were performed using a dual syringe stopped-flow instrument obtained from Hi-Tech Ltd (model SF-51). Measurements were carried out under anaerobic atmosphere, at 25° C. and monitored at 455 nm (an isosbestic point of Compound I and MPO ground state) following rapid mixing of equal volumes of an H₂0₂-containing buffer solution and an MPO solution that contained different NO concentrations. The time course of absorbance change was fit to either single or double exponential functions as indicated. The rate constants for the formation (kₒₙ) and decay (k_{off}) of the MPO-Fe(III)-NO complex in the presence of plasma levels of the alternative substrate Cl⁻ (100 mM) were determined by monitoring absorbance change at 430 nm at 10° C. The time course was accurately fit to the first-order exponential equation (Y=1−e^{-kt}) using a nonlinear least-squares method provided by the instrument manufacturer. Signal to noise ratios for kinetic analyses were improved by averaging at least six to eight individual traces.

Rapid kinetic studies were initially performed under Cl⁻ free conditions using 2 co-substrates, a fixed (low) level of H₂O₂ and variable levels of NO. These conditions were chosen to facilitate the direct examination of NO as a substrate for various forms of MPO in the absence of multiple competing co-substrates. The influence of NO on the kinetics of Compound II buildup, duration, and decay during steady-state catalysis were examined under anaerobic conditions following rapid mixing of enzyme and various concentrations of NO (2.5, 12.5, 50 and 400 μM final) in the presence of physiological concentrations of H₂O₂ (10 μM final). The time course for the formation and decay of compound II in the absence of NO detected by monitoring the absorbance change at 455 nm showed that the change in absorbance that takes place in the first 2 seconds of the reaction and is attributed to the buildup of Compound II. The build-up of Compound II was best fit to a single exponential function, giving an apparent pseudo first-order rate constant of 3.2 s⁻¹. The subsequent decrease in absorbance at 455 nm observed was also fit to a single exponential function with a rate constant of 0.008°-I and was attributed to the decay of compound II. Together, these results indicate that the buildup of MPO Compound II in the absence of NO is rapid, monophasic, and occurs with a much faster rate than its decay.

The addition of NO to reaction mixtures resulted in dramatic effects on the rates of MPO Compound II build-up, duration and decay, as assessed by stopped-flow spectroscopy. (An anaerobic solution containing sodium phosphate buffer (200 mM, pH 7.0) supplemented with H₂O₂ (20 μM) was rapidly mixed with an equal volume of buffer containing 0.86 μM of MPO-Fe(III) and differing concentrations of NO at 25° C.) NO was readily used as a one e⁻ substrate by Compound I, as indicated by the rapid buildup of MPO Compound II. The rate of Compound II accumulation was enhanced nearly 20 fold in the presence of NO and increased in a concentration-dependent and saturable manner. The presence of NO had a variable effect on the duration of steady state concentrations of Compound II that developed following H₂0₂ addition. Finally, NO significantly accelerated the rate of MPO Compound II decay in a concentration-dependent fashion. A plot of NO concentration versus rate of Compound II decay demonstrated linear kinetics and yielded a second order rate constant of 8×10³ M⁻¹S⁻¹.

The accelerated rate of Compound II decay in the presence of NO indicates that it also serves as a one e⁻ substrate for MPO Compound II.

In a parallel series of experiments, the influence of NO on the kinetics of MPO Compound II buildup, duration, and decay during steady-state catalysis in the presence of plasma levels of the competing substrate Cl⁻ was examined. Reactions were again performed under anaerobic conditions and the absorbance change at 455 nm monitored following rapid mixing of enzyme and various concentrations of NO (2.5, 12.5, 25 and 50 μM final) in the presence of H₂0₂ (10 μM final) and Cl⁻ (100 mM final). Anaerobic spectra of MPO-Fe(III) were recorded at 25° C. in septum-sealed quartz cuvettes that were equipped with a quick-fit joint for attachment to a vacuum system. MPO samples were made anaerobic by repeated cycles of evacuation and equilibrated with catalyst-deoxygenated N₂. Cuvettes were maintained under N₂ or NO atmosphere during spectral measurements.

Addition of NO to MPO, H₂O₂ and Cl⁻ resulted in a significant increase in the amount of Compound II formed during steady state catalysis. In the presence of physiologically relevant levels of the competing substrate Cl⁻, stopped-flow analysis of the NO concentration dependence on both the amount of Compound II formed, and the rate of Compound II formation and decay, revealed that NO serves as a substrate for MPO Compounds I and II during steady state catalysis. Finally, to assist in the interpretation of these results, stopped-flow methods were used to determine the association (kₒₙ) and dissociation (k_{off}) rates of NO binding to MPO-Fe(III) in the presence of 100 mM Cl⁻. Analysis of stopped-flow traces collected when the enzyme solutions mixed with NO were accurately fit by a single exponential function. The plots of the apparent rate constants as a function of NO concentration for MPO-Fe(III) were linear (r>0.99), consistent with NO binding to MPO-Fe(III) in a simple one step mechanism. The kₒₙ and k_{off} calculated from the slope and intercept, respectively, for NO binding to MPO-Fe(III) in the presence of 100 mM Cl⁻ at 11° C. were 0.15 μM⁻¹ s⁻¹ and 22.3 s⁻¹, respectively.

### VIII. Nitric Oxide is a Physiological Substrate for EPO

### Example 1

### Tracheal Ring Studies

Normal rats were sacrificed and their trachea removed. Under the dissecting microscope, each trachea was freed of adventitia and fat tissue. A cylindrical airway segment of 3 mm length was isolated from the midtrachea of each animal and placed in a modified Krebs-Henseleit (KH) solution of the following composition (mM): NaCl, 118.2; NaHCO₃, HCl, 4.6; KH₂PO₄, 1.2; MgSO₄, 1.2; CaCl₂ and dextrose, 10%, with a pH adjusted to 7.4. The solution was continuously aerated with 5% C₂ balanced with O₂. Tracheal cylinders were suspended between a sturdy glass rod and a force displacement transducer (FT 03, Grass Instruments, Quincy, Mass.) connected to an amplifier as described in Agani et al. (1997) Am J Physiol 273:L40-L45; and Mhanna et al. (2001) Med J Resp Cell Mol Biol (in press). Generated forces were continuously monitored and recorded on a rectilinear chart recorder. The cylinders were allowed to equilibrate in the organ bath (Radnoti Glass, CA) for 40-45 minutes before challenge. The optical length at which maximal isometric force developed was obtained for each cylinder by 0.1 g increments of load until electrical field stimulation (5 V dc applied through platinum electrodes, 250 mA/cm2) applied for 10 seconds at 4 minute intervals gave a reproducible maximal response.

A cumulative concentration-response curve to bethanecol (3×10⁻⁸ to 10⁻³ M) was obtained for each cylinder. The concentration of bethanecol that elicited 50-75% of maximal response (ED 50-75) was determined. The airway cylinders were then washed, equilibrated, and precontracted with bethanecol (ED 50-75: between 3×10-6 and 10-5 M). Electrical field stimulation (EFS) was then applied to each precontracted cylinder at a range of 0.5, 1, 2, 4, 8, 16, 32, and 64 Hz at a constant voltage (5 V) and dc current. The percent relaxation from the precontracted state was calculated for each cylinder. The percent relaxation in response to EFS was calculated from the reduction in tension (in grams) in relation to baseline tension in the precontracted state, each tracheal segment serving as its own control (before and after EFS exposure). All stock solutions were made freshly on the day of the experiments. Stock solution of oxidases (H₂O₂-generating system) and peroxidases were made in KH buffer. Where indicated, a continuous flux of H₂O₂ was generated with the glucose (0.6 mM final)/glucose oxidase (200 ng/mL) system. Under these conditions, a continuous flux of H₂O₂ is formed (˜1.8 μM/min). Bethanechol was dissolved in distilled water. PGE2 was dissolved in 0.1 M phosphate buffer (pH 7.0) solution.

### Example 2

### Spectroscopic and Rapid Kinetics Characterization of the Interaction Between NO and Compounds I and II of EPO and LPO

To examine the interactions of NO with EPO and LPO the following experiment was performed. Rapid mixing of a solution of EPO-Fe(III) with an equal volume of a 20-fold molar excess of H₂O₂ in the absence of cosubstrates resulted in the rapid formation of a transient complex. The complex displayed a Soret absorbance at 413 nm and broad visible bands centered at 598 and 668 nm, typical of a six-coordinate complex. This spectrum differs from that of either ferric or ferrous EPO, whose Soret maxima are centered at 413 and 450 nm, respectively. The spectrum of the intermediate initially formed following addition of H₂O₂ to EPO-Fe(III) is consistent with formation of EPO compound I (see Furtmuller et al. (2000) Biochemistry 39:15578-15584) and similar to that of LPO and MPO compound I (see Burner et al. (2000) J Biol Chem 275:20597-20601; Marquez et al. (1995) J Biol Chem 270:30434-30440; Kimura et al. (1979) Arch Biochem Biophys 198:580-588; and Marquez et al. (1994) Biochemistry 33:1447-1454). This EPO intermediate formed within 50 ms after mixing at 25° C. but was unstable and rapidly converted into a more stable intermediate within 3 second, as judged by a time-dependent shift in Soret absorbance from 413 to 432 nm. This species displayed a Soret band at 432 nm and two visible bands at 532 and 564 nm, consistent with formation of EPO compound II. EPO compound II was also unstable and converted gradually to the ground state, EPO-Fe(III), within minutes of initiating the reaction. Spectral transitions between each intermediate formed revealed distinct and well-defined isosbestic points. Thus sequential formation and decay of EPO intermediates within the peroxidase cycle occurred at sufficiently different rates to enable each process to be studied by conventional, i.e. single mixing stopped-flow methods. Addition of H₂O₂ to LPO-Fe(III) in the absence of cosubstrates similarly leads to the accumulation of compound II in the millisecond time frame via transient initial formation of compound I and subsequent spontaneous 1e⁻ heme reduction, similar to that observed with EPO and MPO (see Furtmuller et al. (2000) Biochemistry 39:15578-15584; Burner et al. (2000) J Biol Chem 275:20597-20601; Marquez et al. (1995) J Biol Chem 270:30434-30440; Kimura et al. (1979) Arch Biochem Biophys 198:580-588; and Marquez et al. (1994) Biochemistry 33:1447-1454).

Stopped-flow spectroscopy was used to investigate how NO interacts with intermediate forms of EPO and LPO during steady-state catalysis. The influence of NO on the kinetics of EPO and LPO compound II buildup, duration, and decay was examined under anaerobic conditions following rapid mixing of enzyme and various concentrations of NO in the presence of physiological concentrations of H₂O₂ (10 μM final). The time courses for the formation and decay of compound II of both EPO and LPO in the absence of NO were examined by monitoring the absorbance change at 445 nm for EPO and at 430 nm for LPO. For both enzymes, the changes in absorbance that took place in the first 2 seconds of the reactions are shown and are attributed to the buildup of compound II. The buildup of compound II of both EPO and LPO was best fit to a single-exponential function, giving apparent pseudo-first-order rate constants of 4.3 s⁻¹ and 4.6 s⁻¹, respectively. The subsequent decreases in absorbance at 455 and 430 nm observed for both enzymes were also fit to a single-exponential function with a rate constant of 0.005 s⁻¹ for EPO and 0.0005 s⁻¹ for LPO. These spectral changes were attributed to the decay of compound II of EPO and LPO. Together, these results indicate that the buildup rates of compound II formation for each enzyme in the absence of NO is rapid, monophasic, and occurs with a much faster rate than its decay.

The addition of NO to reaction mixtures of EPO or LPO results in dramatic effects on the rates of compound II buildup, duration, and decay, as assessed by stopped-flow spectroscopy. For both enzymes, NO was readily used as a 1e⁻substrate by compound I, as indicated by the enhanced rate of compound II formation. For EPO, the rate of compound II accumulation was enhanced nearly 25-fold in the presence of NO and increased in a concentration-dependent and saturable manner. In contrast, the rate of LPO compound II accumulation increased in a linear manner as a function of NO concentration, yielding a second-order rate constant of 2×106 M-1 s-1. In addition, NO influenced the steady-state level of EPO and LPO compound II formed following H₂O₂ addition. As the concentration of NO present in reaction mixtures was increased, the steady-state levels of LPO compound II generated progressively decreased, as judged by the decrease in overall absorbance at 430 nm developed during steady-state catalysis. The presence of NO had a variable effect on the duration of maintaining steady-state concentrations of compound II for each peroxidase following H₂O₂ addition. Finally, NO significantly accelerated the rate of compound II decay for EPO and LPO in a concentration-dependent fashion. Plots of NO concentration versus observed rates of compound II decay for each peroxidase demonstrated linear kinetics and yielded second-order rate constants of 1.7×104 M⁻¹ s⁻¹ and 8.7×104 M⁻¹ s⁻¹ for EPO and LPO, respectively. The accelerated rate of compound II decay in the presence of NO indicates that NO also serves as a 1e⁻ substrate for both EPO compound II and LPO compound II.

### Example 3

### NO Is Catalytically Consumed by Peroxidases Enriched in Asthmatic Airways Under Physiological Conditions

Using an NO-selective electrode, the following study was performed to show that catalytic amounts of EPO or LPO could accelerate NO consumption in the presence of O₂.— and plasma levels of halides. In both cases, the NO levels used were much less than the K_{diss} (k₋₁/k₁) for LPO or EPO Fe(III)-NO (see Abu-Soud et al. (2001) Biochemistry (in press)), which limits the interaction between NO and the enzymes. In control reactions lacking O₂^{.-} generating system, neither peroxidases nor H₂O₂ alone significantly influenced the removal of NO. The present studies thus suggest a functional role for peroxidases as catalytic sinks for NO in inflamed airways.

### Example 4

### Peroxidase/H₂O₂ Systems Reversibly Attenuate NO-Dependent Bronchodilation of Tracheal Rings

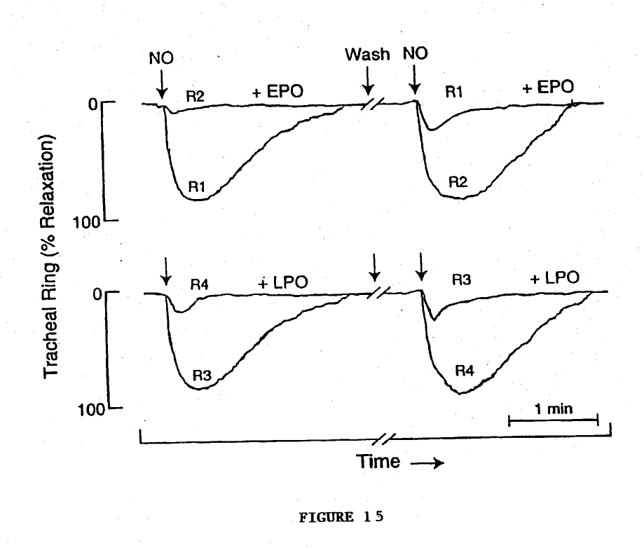

To examine the potential physiological relevance of peroxidase-NO interactions, the capacity of EPO, LPO, and MPO to attenuate NO-dependent relaxation of tracheal rings was examined. To determine whether catalytic levels of peroxidases (e.g., data for EPO/H₂O₂ and LPO/H₂O₂ systems shown) inhibit NO-dependent bronchodilation, precontracted tracheal rings were used. Briefly, different tracheal rings (FIG. 15, data for four rings, R1-R4, shown) were isolated and precontracted with bethanecol (a long-acting cholinergic agent) that evoked a force of contraction of approximately 1 g. All rings were incubated in organ bath media in the presence of a slow flux of H₂O₂ (˜1.8 μM/minute) generated by the glucose/glucose oxidase system as described. Control experiments demonstrated that the H₂O₂-generating system had no significant effect on NO-dependent bronchodilation of the preconstricted rings. Before NO exposure, R2 and R4 were treated with catalytic amounts of either EPO or LPO, while the control rings, R1 and R3, remain untreated. Addition of NO to precontracted tracheal rings in the absence of peroxidases (R1 and R3) resulted in the anticipated time-dependent bronchodilatory responses. In contrast, pretreatment of rings with catalytic levels of peroxidases (EPO and LPO, shown in R2 and R4 of FIG. 15, respectively) dramatically attenuated the normally observed bronchodilation that follows addition of NO. Following a wash out period, rings previously treated with peroxidases (R2 and R4) again demonstrated NO-dependent bronchodilatory responses, confirming that the NO-dependent signaling pathways in the previously treated rings were still intact. Taken together, these experiments demonstrate that mammalian peroxidases and H₂O₂, both enriched in asthmatic airways, can prevent NO-dependent bronchodilatory responses.

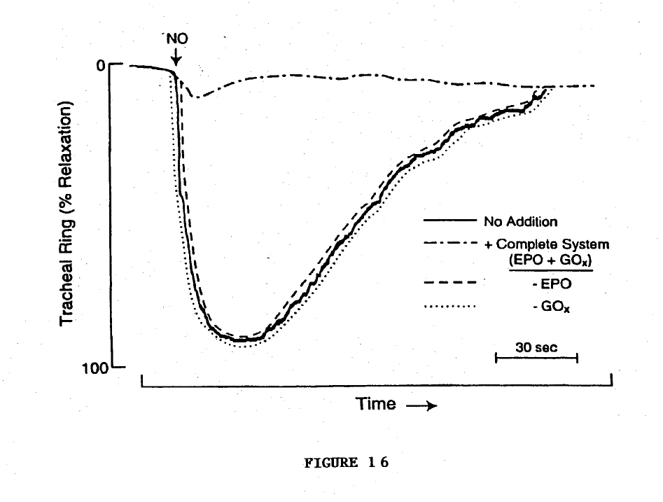

To further characterize the capacity of peroxidase-H₂O₂ systems to inhibit NO-mediated relaxation of tracheal rings, the reaction requirements necessary for peroxidase-dependent inhibition of NO-mediated bronchodilatory responses were examined. As shown in FIG. 16, NO-mediated relaxation of tracheal rings occurred in the presence of either peroxidase or an H₂O₂-generating system but not the combined presence of both peroxidase and H₂O_{2.}

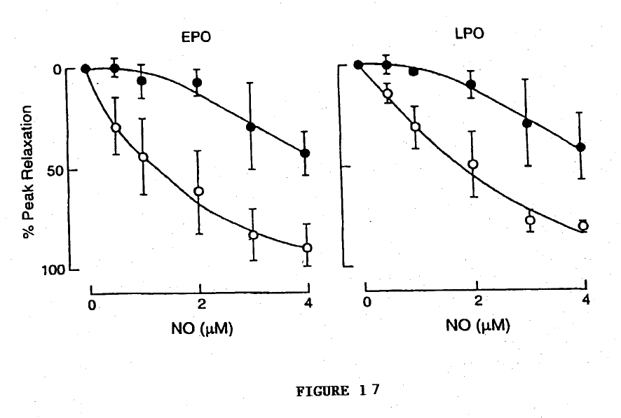

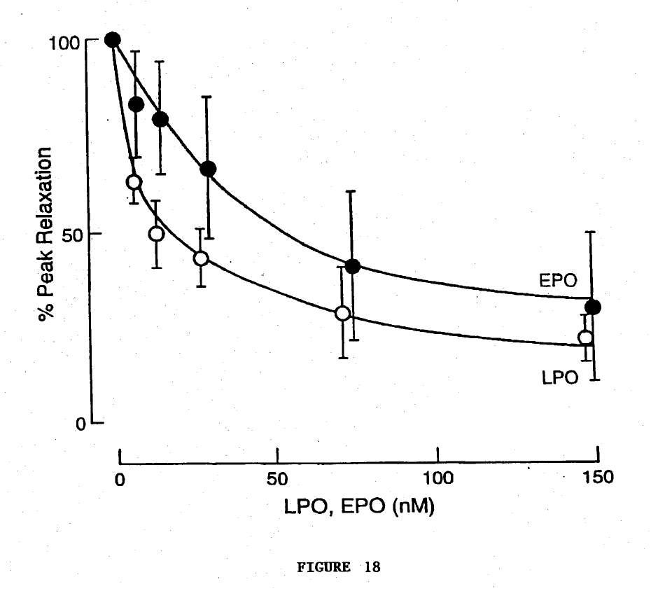

The effect of peroxidases on the concentration dependence of NO-mediated relaxation of preconstricted tracheal rings was quantified. Experiments were performed by equilibrating preconstricted tracheal rings in Krebs buffer supplemented with glucose/glucose oxidase (a H₂O₂-generating system; ˜1.8 μM/minute) and then assessing ring relaxation in response to varying amounts of NO in the absence and presence of specific peroxidases (EPO, LPO, or MPO). Typical NO-dependent relaxation curves of tracheal rings in the presence and absence of EPO and LPO are illustrated in FIG. 17. In the absence of peroxidases, relaxation of the organ rings increased as a function of NO concentration and reached a maximum at ˜4 μM NO. In contrast, incubation of the tracheal rings with the bath media containing catalytic levels of peroxidase (data for EPO and LPO shown) attenuated NO-dependent relaxation, as reflected in the rightward shift of the dose-response curve. Finally, the concentration dependence of peroxidase-mediated inhibition of NO-triggered bronchodilation was assessed using preconstricted rat tracheal rings incubated with increasing concentrations of peroxidase in the presence of a fixed low flux (˜1.8 μM) of H₂O₂ (FIG. 18). Tracheal ring relaxation was achieved by exposing rings to fixed amounts of NO (15 μM). Increasing concentrations of peroxidase (data for EPO and LPO shown) significantly attenuated NO-mediated bronchodilation and reduced the maximal response to NO (FIG. 18). Collectively, these results are consistent with peroxidases serving as a catalytic sink for NO, preventing tracheal relaxation.

### Example 5

### Conclusion

Peroxidases may serve to modulate NO bioavailability and function by serving as a catalytic sink for NO. Peroxidases directly promote NO consumption by using it as al e⁻ substrate. Peroxidases may also indirectly promote NO consumption through generation of free radical species, which scavenge NO. Alternatively, NO may serve to modulate peroxidase catalytic activity. When serving as a substrate, NO may enhance catalytic rates by reducing compound II to form ferric enzyme, the rate-limiting step in the peroxidase cycle. NO may also promote alterations in substrate selectivity by influencing the distribution of peroxidase intermediates capable of executing 1e⁻ versus 2e⁻ oxidation reactions. Finally, NO may serve as a ligand for ferric peroxidases generating inactive Fe(III)-NO complexes.

### IX. EOS are a Major Source of Nitric Oxide-Derived Oxidants in Severe Asthma

### Example 1

### General Materials

All solvents were purchased from Fisher Scientific (Pittsburgh, Pa.) and were Optima or HPLC grade. PAPA NONOate (Z)-[N-(3-aminopropyl)-N-(n-propyl)amino]diazen-1-ium-1,2-diolate was obtained from Alexis (San Diego, Calif.). Glucose oxidase (grade II) and catalase were acquired from Roche Molecular Biochemicals (Indianapolis, Ind.). L-L⁻¹³C₉¹⁵N]tyrosine and L-[¹³C₆]tyrosine were obtained from Cambridge Isotope Laboratories (Andover, Mass.). Unless otherwise stated, all other chemicals were purchased from Sigma (St. Louis, Mo.).

### Example 2

### Subjects and Sample Collection

Human endotracheal/bronchial aspirates were obtained from patients (n=11) who required mechanical ventilatory assistance due to respiratory failure from a severe asthmatic exacerbation. All asthmatic subjects had a history of a >12% increase in FEV₁ and a 200-cc increase in forced expiratory volume either spontaneously or after bronchodilator within 1 year of hospitalization, and satisfied the definition of severe asthma as defined by the National Institutes of Health guidelines (National Heart, Lung and Blood Institute. Expert Panel Report 2: Guidelines for the Diagnosis and Management of Asthma. National Institutes of Health, Bethesda, Md., Publication No. 97-4051, p. 86.). Samples were collected within 12 hours of admission because asthmatic subjects received i.v. corticosteroids upon presentation to the emergency department and subsequent transfer to the intensive care unit, and the effects of corticosteroids on protein oxidation products is unknown. All asthmatic individuals had a history of using inhaled β2-agonists either regularly (n=8) or on an as-needed basis (n=3) in the month preceding admission. Several also used inhaled corticosteroids (n=8) and/or had received oral corticosteroids within 1 month of presentation (n=4). Control subjects (n=12) were age- and sex-matched, nonsmokers, and had no prior history of asthma or other lung disease. Endotracheal/bronchial aspirates were obtained from several control subjects who were either undergoing elective surgery (n=3) or were admitted to the intensive care unit for a noninflammatory, nonrespiratory process (i.e., airway protection secondary to either head trauma (n=2) or drug overdose (n=2)). The remaining clinical specimens from healthy nonasthmatic controls (n=5) were obtained as residual specimens collected as baseline BAL samples for a separate clinical study. Healthy control subjects in that study all had a negative methacholine challenge test. Thus, all 11 specimens from asthmatic subjects were obtained as endotracheal/bronchial aspirates, and the 12 specimens from nonasthmatic controls were obtained as endotracheal/bronchial aspirates (n=7) and baseline BAL (n=5) specimens. No differences were noted in levels of NO₂—Y, Br—Y, or 3-Cl—Y (per mol tyrosine) in endotracheal/bronchial aspirates vs BAL; consequently, specimens from the nonasthmatic subjects were combined as a single control group. Cells in clinical specimens were removed by centrifugation. Cell supernatants were supplemented with a mixture of antioxidants and peroxidase inhibitors (final concentration: 200 μM diethylenetriaminepentaacetic acid (DTPA), 100 μM butylated hydroxytoluene, 50 mM sodium phosphate (pH 7.0), 1 mM aminotriazole) and then capped in vials under argon atmosphere. Supernatants were then snap frozen in liquid N₂, and stored at −80° C. until time of sample preparation and mass spectrometry analysis.

### Example 3

### Histological Analysis of Lung and Bronchial Tissues

Histological sections were cut from paraffin blocks of lung and bronchial tissues from four individuals who died from asthma and four nonasthmatic age-matched individuals who died of nonpulmonary processes. For NO₂—Y immunostaining, slides were initially incubated at 37° C. with 0.01 mg/mL protease K for 15 minutes. Following wash with PBS containing 0.5 mM levamisole, the tissue was treated with 1% BSA in PBS to block nonspecific binding, then incubated for 2 hours with immunopurified polyclonal Ab directed against NO₂—Y (1:150 diluted in 1% BSA/PBS; Upstate Biotechnology, Lake Placid, N.Y.). Following washing with PBS/0.5 mM levamisole, the tissue was incubated with a biotin-conjugated secondary Ab (Dako, Carpinteria, Calif.) for 10 minutes. Washing was followed by another 0-minute incubation with alkaline phosphatase-labeled streptavidin (Dako). Immunostaining was visualized with an alkaline phosphate substrate solution containing napthol AS-MX phosphatase, Fast Red, and levamisole in Tris buffer (pH 8.2) (Dako) and counterstained with the nuclear stain, hematoxylin. Negative control experiments involved either immunoabsorption of NO₂—Y Ab with 3.75 mM NO₂—Y before incubation with tissue sections or incubating the section with isotype-control nonimmune Ig instead of the primary Ab. In separate studies, the specificity of the primary Ab was confirmed by observing loss of NO₂—Y-specific recognition following reduction of NO₂—Y-containing protein with dithionite. All biopsies were stained on the same day. Specific autofluorescence imaging of EPO in biopsies was performed as described in Fuerst et al. (1965) Nature 205:1333; Weil et al. (1981) Blood 57:1099; and Samoszuk et al. (1987) Blood 70:597). EPO-specific in situ peroxidase staining of tissues was performed on anti-NO₂—Y-stained tissue sections following treatment of slides with 0.01 M KCN to inhibit MPO (Bos et al. (1981) Infect Immun 32:427; Samoszuk et al. (1986) Am J Pathol 125:426.).

### Example 4

### EO and Neutrophil Isolation

Human EOs were isolated by negative selection using CD16 microbeads (Miltenyi Biotec, Auburn, Calif.) as described in Hansel et al. (1989) J Immunol Methods 122:97. Human neutrophils were isolated by buoyant density centrifugation (Hazen et al. (1996) J Biol Chem 271:1861), and low levels of contaminating EOs were then removed by fluorescence-activated cell sorting as described in Thurau et al. (1996) Cytometry 23:150. The final purity of cell preparations was confirmed by flow cytometry using selective Abs for cell surface Ag on EOs (CD49d) and neutrophils (CD16), respectively (Thurau et al. (1996) Cytometry 23:150). No detectable cross-contamination of peroxidase activity in detergent extracts of leukocyte preparations was observed following SDS-PAGE with in-gel tetramethylbenzidine peroxidase staining (see van Dalen et al. (1997) Biochem J 327:487). Trypan blue exclusion tests demonstrated over 97% viability in EO and neutrophil preparations.

### Example 5

### Cell Experiments

These studies were performed in the presence of CO₂ (5% gas phase) and HCO₂—n the medium (4.2 mM) to mimic a biologically relevant situation. Leukocytes (1×1×10⁶/mL) were incubated at 37° C. under 95% air, 5% CO₂, in Medium A (Ca²⁺/Mg²⁺/phenol-free HBSS; Life Technologies, (Gaithersburg, Md.) supplemented with 100 μM NaBr, 50 μM L-arginine, and 200 μM DTPA, pH 7.4 final) in the absence or presence of NaNO₂ (0<50 μM, as indicated in the figure legends). Where indicated, 100 μM of either L-tyrosine or its deaminated analog, 3-(4-hydroxyphenyl)propanoic acid (HPA) was included. Cells were activated by addition of PMA (200 nM) and incubated for either 1 hour or the indicated time interval. In some experiments, EOs were activated in the presence of an exogenous NO source by addition of PAPA NONOate (Alexis) for 1 minute following PMA addition. Rates of NO flux were determined spectrophotometrically by reaction of NO with oxyhemoglobin as described in Baek et al. (Baek et al. (1993) J Biol Chem 268:21120) under the identical conditions used for experiments, but in the absence of any added cells. To maintain a final pH of 7.4 during experiments with PAPA NONOate, incubations were performed in Medium B (Medium A containing only 100 mM NaCl and supplemented with 20 mM sodium phosphate, pH 7.4). NO₂⁻ levels accumulated to over 50 μM in medium incubated with the NO-generating system (2 μM/min) in the absence of cells. In some cases, leukocyte reaction mixtures also contained one of the following: 1 mM NaN3, 10 mM 3-aminotriazole (Atz), 300 nM catalase (Cat), 300 nM heat-inactivated catalase (hiCat), 10 μg/ml superoxide dismutase (SOD), 1 mM methionine (Met), or 1 mM N^{α}-acetyl lysine.

### Example 6

### Quantification of Leukocyte-Generated Products In Vitro

NO₂—Y production by isolated EOs was quantified by reversed phase HPLC with photodiode array detection (see Wu et al. (1999) J Biol Chem 274:25933). Peak identity was routinely confirmed by demonstrating the appropriate UV-VIS absorbance spectrum of the peak that comigrated with authentic NO₂—Y. In preliminary studies, NO₂—Y production by EOs was also independently confirmed by HPLC with on-line electrospray ionization mass spectrometry, similar to prior studies using isolated EPO. The nitrated (NO₂-HPA; 3-(4-hydroxy-3-nitrophenyl)propanoic acid), brominated (Br-HPA; 3-(3-bromo-4-hydroxyphenyl)propanoic acid), and chlorinated (Cl-HPA; 3-(3-chloro-4-hydroxyphenyl)propanoic acid) products of the tyrosine analog, HPA, were routinely quantified by reversed phase HPLC with electrochemical (coulometric) detection on an ESA CoulArray HPLC (Cambridge, Mass.) equipped with UV detector and electrochemical cells (eight channels) (Wu et al. (1999) J Biol Chem 274:25933). Peak identity was established by demonstrating the appropriate retention time, redox potential, ratio of integrated currents in adjacent channels, and by the method of standard additions for each analyte. Authentic standards of NO₂-HPA, Br-HPA, and Cl-HPA were prepared by reaction of HPA with a molar equivalent of ONOO⁻, HOBr, or HOCl, respectively. Standards were then isolated by reversed phase HPLC, and their structures were confirmed by electrospray ionization mass spectrometry by demonstrating the appropriate mass-to-charge ratio (and isotopic cluster, where applicable) of the anticipated molecular ion of the isolated product.

### Example 7

### Sample Preparation and Mass Spectrometry

The contents of Br—Y and Cl—Y in proteins present in clinical specimens were determined by stable isotope dilution GC-MS using 3-bromo[¹³C₆]tyrosine and 3chloro[¹³C₆]tyrosine as internal standards. The NO₂—Y content of lavage proteins was quantified by stable isotope dilution GC-MS following reduction to aminotyrosine (see Crowley et al. (1998) Anal Biochem 259:127). All results were normalized to the content of the precursor amino acid, L-tyrosine, which was similarly quantified by GC-MS (58) using L-[¹³C₉¹⁵N]tyrosine as internal standard. Intrapreparative formation of 3-bromo[¹³C₉¹⁵N]tyrosine, 3-chloro[¹³C₉¹⁵N]tyrosine, or 3-nitro[¹³C₉¹⁵N]tyrosine was routinely monitored and found to be negligible (i.e., <5% of the level of the natural abundance product observed) under the conditions used.

### Example 8

### General Procedures

All water used to prepare buffers and medium was pretreated with Chelex-100 resin (Bio-Rad, Hercules, Calif.) and supplemented with 100 μM DTPA to remove trace levels of potential redox-active transition metal ions. Superoxide generation by activated human EOs was measured as the SOD-inhibitable reduction of ferricytochrome c (Babior et al. (1973) J Clin Invest 52:741). Quantification of NO₂— and NO₃— was performed by anion exchange HPLC with UV detection at 210 nm under argon atmosphere. Products were resolved on a Spherisorb S5 SAX column (24 cm×4.6 mm, 5 μm; Phase Separations, Norwalk, Conn.) under isocratic conditions using 45 mM sodium phosphate (pH 3.0) as the mobile phase.

Leukocytes were isolated from whole blood of healthy volunteers after obtaining informed consent. Tissue sections were obtained from the New Mexico Office of the Medical Examiner and the Anatomic Pathology Department at the Cleveland Clinic Foundation. Sections were anonymized for use in these studies.

### Example 9

### Statistics

Data represent the mean±SD of the indicated number of samples. Statistical analyses were made using a paired Student's t test. For all hypotheses the significance level was 0.05. When multiple comparisons were made, a Bonferroni correction to the significance criterion for each test was made.

### Example 10

### RNS Contribute to Oxidative Modification of Proteins in Asthma

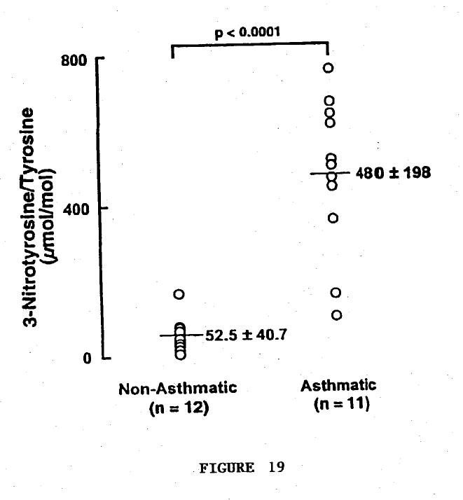

To quantify the potential role of RNS in promoting protein oxidation in asthma, a stable isotope dilution GC-MS was used to compare the protein content of NO₂—Y recovered from airways of subjects with severe asthma vs non-asthmatic subjects. A significant (p<0.0001) 10-fold increase in protein NO₂—Y levels was observed in samples from severe asthmatic patients compared with levels present in non-asthmatic subjects, see FIG. 19 (480±198 vs 52.5±40.7 μmol tyrosine; asthmatic vs non-asthmatic, respectively).

### Example 11

### Immunohistochemical Studies Colocalize NO₂—Y With EOs in Bronchial Tissues From severe Asthmatics

To identify the potential cellular source(s) of NO-derived oxidants in severe asthma, specimens from subjects who died from asthma (status asthmaticus) were examined using affinity-purified Ab specific for NO₂—Y.

Lung and bronchial tissue from individuals who died of asthma were sequentially immunostained for NO₂—Y followed by cytochemical staining for EPO. Results indicated colocalization of EPO and NO₂—Y in the submucosa of bronchial tissues from distinct subjects with status asthmaticus. Intense NO₂—Y staining was predominantly observed colocalizing within intact EOs, which was recognizable by their characteristic bilobed nuclei. The strong red staining for NO₂—Y obscured the cytoplasmic staining for EPO in these EOs.

In general, intense staining that colocalized with EOs was typically observed in the majority of specimens. Diffuse staining of epithelial cells was also commonly observed. Both in situ fluorescence microscopy specific for the heme group of EPO and in situ peroxidase staining specific for EPO were also abundant in EO-rich areas of specimens from asthmatics. Double staining of sections for both NO₂—Y — and EPO-specific in situ peroxidase staining confirmed colocalization of NO₂—Y with EOs in the submucosa of airways from severe asthmatic subjects.

### Example 12

### EOs are a Major cellular Mediator of Protein Oxidation in Severe Asthma

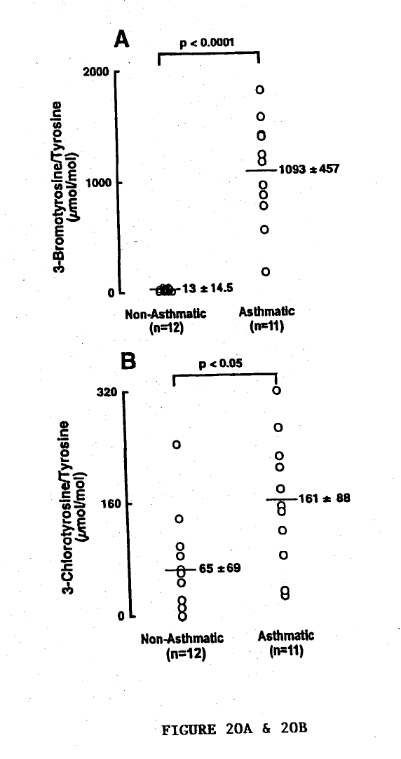

To assess the relative contributions of EOs and neutrophils in the oxidative modification of proteins in severe asthma, the protein content of Br—Y and Cl—Y, molecular fingerprints for EO- and neutrophil-mediated tissue damage, respectively, were determined in the same clinical specimens evaluated for protein NO₂—Y content in FIG. 19. There was a striking 84-fold elevation (p<0.0001) in the content of Br—Y observed in proteins recovered from airways of asthmatic (1093±457 μmol Br—Y/mol tyrosine) vs nonasthmatic subjects, whose levels were near the limit of detection (13±14.5 μmol Br—Y/mol tyrosine) (FIG. 20A). There was also a significant 3-fold increase (p<0.05) in Cl—Y in airway proteins recovered from severe asthmatics (161±88 μmol Cl—Y/mol tyrosine) over nonasthmatics (65±69 μmol Cl—Y/mol tyrosine) (FIG. 20B). A comparison of the Br—Y/Cl—Y ratios, an indication of the relative preferential contribution of EOs vs neutrophils toward oxidation of proteins, revealed a 30-fold difference in asthmatics compared with nonasthmatics (ratio of 6.8 versus 0.2 for asthmatic versus nonasthmatic, respectively).

### Example 13

### Human EOs Nitrate Tyrosine in the Presence of Physiological Levels of Halides and NO₂—

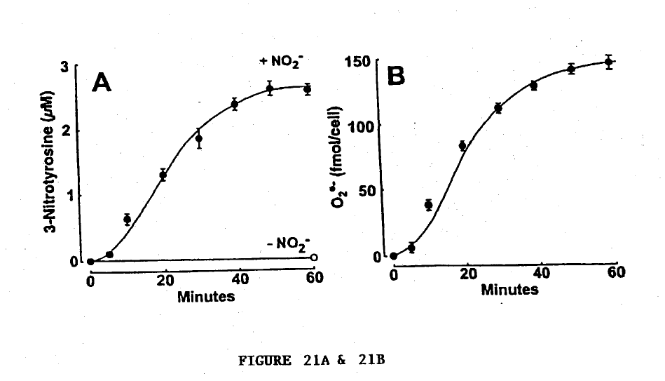

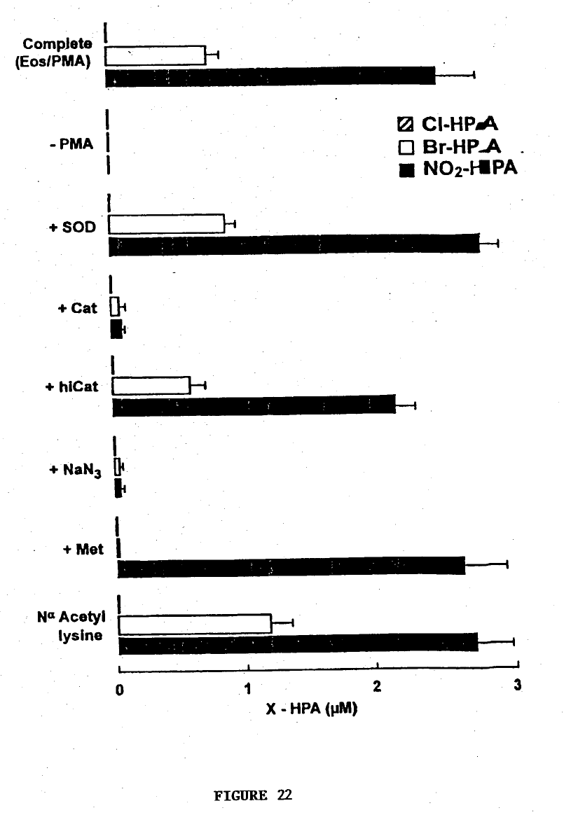

The following experiment was performed to determine if EOs use EPO to contribute to protein oxidation through nitration in asthma. To determine whether EOs can generate NO-derived oxidants, peripheral cells from normal healthy donors was isolated and incubated them in medium containing L-tyrosine, plasma levels of halides (Medium A), and the agonist PMA. Analysis of medium revealed that no significant NO₂—Y was formed (FIG. 21A).

Moreover, no endogenous NO production or significant (i.e., >1 μM) NO₂—/NO₃— accumulation by human EOs freshly isolated from peripheral blood was detected with or without phorbol ester activation, under the conditions and time course used. In contrast, EOs activated in Medium A supplemented with pathophysiologically relevant levels of NO₂⁻ (50 μM) readily produced NO₂—Y (FIG. 21A). The time course for NO₂—Y formation paralleled the time course for O₂^{.-} production during a respiratory burst (FIG. 21B).

Finally, in separate studies, EOs were activated with an alternative agonist, N-formyl-methionyl-leucyl-phenylalanine (100 nM). Cell-dependent NO₂—Y formation again demonstrated an absolute requirement for exogenous NO₂⁻, although NO₂—Y levels produced were 8-fold less than that observed with EOs stimulated with PMA.

The absolute requirement of NO₂⁻ for NO₂—Y formation suggested that, under conditions used, EOs were generating RNS via the EPO-H₂O₂—NO₂— system. To further explore the reaction mechanism for EO-mediated aromatic nitration reactions, isolated human EOs were incubated in medium containing plasma levels of halides (100 mM Cl⁻, 100 μM Br⁻), levels of NO₂⁻ observed in epithelial lining fluid of severe asthmatics (50 μM), and the deaminated analog of tyrosine, 3-(4-hydroxyphenyl)propanoic acid (HPA, 100 μM). Use of this low molecular mass surrogate for a protein-bound tyrosine residue permitted more facile quantification of the relative capacity of EOs to promote aromatic nitration and halogenation reactions of phenolic targets.

The capacity of EOs to promote aromatic nitration, bromination, or chlorination reactions was assessed by quantifying production of NO₂-HPA, Br-HPA, and Cl-HPA, respectively. Following activation by PMA (Complete System, FIG. 22) EOs preferentially nitrated HPA, generating 3-fold more NO₂-HPA than Br-HPA, and no detectable Cl-HPA (FIG. 22).

EO-mediated nitration required cell activation and was inhibited by either the presence of the hydrogen peroxide scavenger catalase (but not hiCat) or peroxidase inhibitors (e.g. NₐN₃). In contrast, nitration reactions were not blocked by the presence of O₂^{.-}-scavengers (SOD) or scavengers of hypohalous acids (FIG. 22). Similar reaction requirements were observed for EO-mediated bromination reactions (i.e., sensitivity to H₂O₂ scavengers and peroxidase inhibitors) (FIG. 22). One distinguishing feature between EO-mediated nitration and bromination reactions is the inability of the HOBr scavenger methionine to affect nitration by the cells. The lack of inhibition in cell-dependent bromination observed in the presence of the primary amine-containing species, N-acetyl lysine, is consistent with prior observation that N-bromoamines serve as excellent mediators of aromatic bromination reactions.

### Example 14

### EOs are More Efficient Than Neutrophils at Promoting Aromatic Nitration Reactions

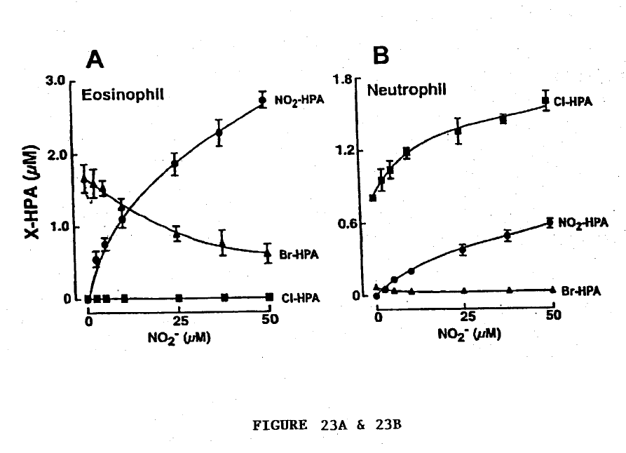

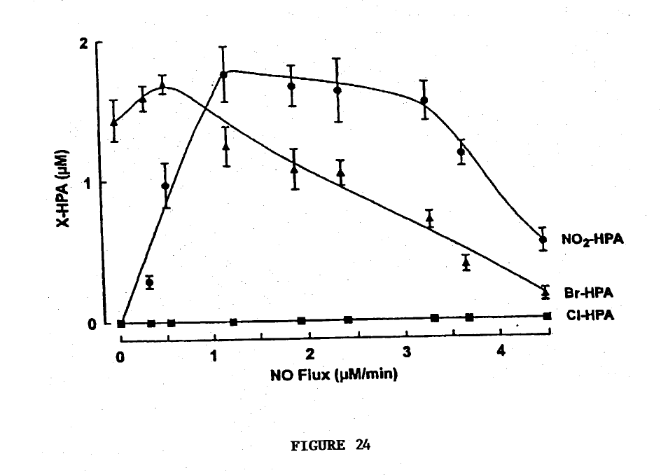

In the next series of experiment the ability of purified human EOs and neutrophils to nitrate, chlorinate, and brominate the tyrosine analog HPA in the presence of plasma levels of halides over the (patho)-physiologically relevant range of NO₂⁻ (0-50 μM) was compared. EOs generated significantly more NO₂-HPA (>5-fold) than an equivalent number of neutrophils at all concentrations of NO₂⁻ examined (FIG. 23). At levels of NO₂— observed in epithelial lining fluid from normal individuals (i.e. <10 μM NO₂⁻), EOs were more effective at oxidation of phenolic groups through bromination. As NO₂⁻ levels became elevated into the pathophysiological range (>10 μM), nitration of targets predominated (FIG. 23A). In stark contrast to neutrophils, no significant oxidation of HPA through chlorination was observed under all conditions examined (FIG. 23). Finally, neutrophils were ineffective at oxidizing the tyrosine analog through bromination under all conditions examined (FIG. 23).

### Example 15

### EOs Generate NO-Derived Oxidants by at Least Two Mechanistically Distinct Pathways

One striking feature of the results thus far described was that there were no evidence that isolated EOs generate nitrating intermediates through formation of ONOO⁻. Based upon the inability to detect NO₁, NO₂—, or NO₃— accumulation in cell medium, this likely reflects the limited capacity of freshly isolated peripheral blood EOs from healthy (nonallergic) donors to generate NO, particularly within the brief time period of the respiratory burst (˜1 hour, FIG. 21B). A series of experiments in which EOs were activated in the presence of an exogenous NO-generating system, PAPA NONOate, and plasma levels of Br⁻ (100 μM) was performed. These conditions should more closely mimic a physiological mechanism for NO₂⁻ formation, as well as provide an environment where EO-generated O₂^{.-} might react with NO before it dismutates into the EPO substrate, H₂O₂. Although osinophils activated in the absence of the NO donor failed to mediate nitration reactions, cells stimulated in the presence of a continuous NO source readily formed NO₂-HPA (FIG. 24). Aromatic nitration by EOs required cell activation, consistent with a requirement for reduced oxygen species (O₂^{.-} and/or H₂O₂) for oxidation. Moreover, nitration was the favored biochemical pathway for oxidative modification at all but the lowest levels of NO flux examined. At the higher fluxes of NO (>2 μM/min) examined, the overall extent of NO₂-HPA formation diminished (FIG. 24).

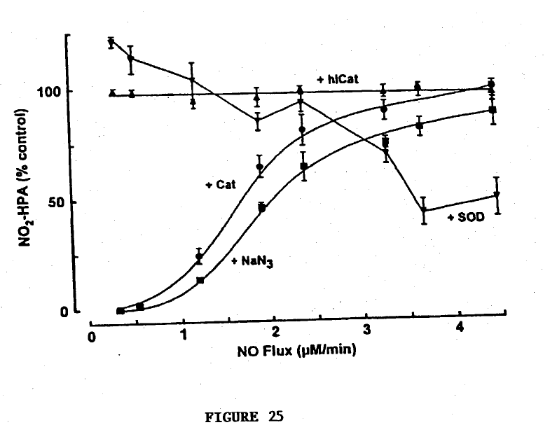

Aromatic nitration reactions mediated by EOs in the presence of an exogenous NO donor might occur via formation of either ONOO— (and/or ONOOCO₂⁻) or the EPO-H₂O₂—NO₂— system. To gain insights into the pathway(s) used by EOs to generate NO-derived oxidants, the effects of EPO inhibitors, H₂O₂ scavengers, and SOD on NO₂-HPA formation were examined. At low rates of NO flux (<2 μM/min), NO₂-HPA formation was inhibited by azide, a heme poison that inhibits EPO catalysis (FIG. 25). Under these same conditions, catalase, but not hiCat, significantly attenuated NO₂-HPA production, consistent with a role for H₂O₂ in the aromatic nitration reaction. In contrast, at high rates of NO flux (>2 μM/min), aromatic nitration by activated EOs became increasingly less sensitive to inhibition by either azide or catalase. Finally, while addition of SOD to reactions demonstrated a modest increase in NO₂-HPA formation at low levels of NO flux, inhibition in nitration was observed at the higher rates of NO flux examined (FIG. 25).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating an eosinophil-mediated inflammation in a mammal, said method comprising administering a pseudohalide to said mammal under conditions such that said eosinophil-mediated inflammation is reduced.

2. The method of claim 1, further comprising monitoring the level of said pseudohalide in said mammal.

3. The method of claim 1, wherein said eosinophil-mediated inflammation is selected from the group consisting of asthma, rhinitis, eczema, contact dermatitis, hypereosinophilic syndrome, and polyposis.

4. The method of claim 1, wherein said pseudohalide has an oxidative reactivity with a peroxidase greater than that of bromide.

5. The method of claim 1, wherein said pseudohalide, when oxidized by eosinophil peroxidase, forms a product having lower toxicity than HOBr.

6. The method of claim 1, wherein said pseudohalide is administered orally, nasally, or topically.

7. The method of claim 1, wherein said pseudohalide is administered by injection or inhalation.

8. The method of claim 1, wherein said administration comprises administering a pill or a dietary supplement containing said pseudohalide.

9. The method of claim 1, wherein said pseudohalide is administered at a dose between 1 μg to 10 g/day.

10. A method of reducing peroxidase catalyzed oxidation of a halide in a mammal, said method comprising administering a pseudohalide to said mammal such that said pseudohalide contacts said peroxidase and reduces the oxidation of said halide.

11. The method of claim 10, further comprising monitoring the level of said pseudohalide in said mammal.

12. The method of claim 10, wherein said peroxidase is selected from the group consisting of eosinophil peroxidase, myeloperoxidase, and lactoperoxidase.

13. The method of claim 10, wherein said halide is selected from the group consisting of bromide, iodide, and chloride.

14. The method of claim 10, wherein said mammal is a human.

15. The method of claim 10, wherein said pseudohalide is selected from the group consisting of thiocyanate, salts of thiocyanate, isothiocyanate, and salts of isothiocyanate.

16. A method for treating a mammal having bronchial constriction, said method comprising administering a pseudohalide to said mammal such that said bronchial constriction is reduced.

17. The method of claim 16, further comprising monitoring the level of said pseudohalide in said mammal.

18. The method of claim 16, wherein said mammal is a human.

19. The method of claim 16, wherein said pseudohalide is selected from the group consisting of thiocyanate, salts of thiocyanate, isothiocyanate, and salts of isothiocyanate.

20. The method of claim 16, wherein said pseudohalide has an oxidative reactivity with a peroxidase greater than that of bromide.

21. The method of claim 16, wherein said pseudohalide, when oxidized by eosinophil peroxidase, forms a product having lower toxicity than HOBr.

22. The method of claim 16, wherein said pseudohalide is administered orally, nasally, or topically.

23. The method of claim 16, wherein said pseudohalide is administered by injection or inhalation.

24. The method of claim 16, wherein said administration comprises administering a pill or a dietary supplement containing said pseudohalide.

25. A method for increasing the amount of NO in the tissue of a mammal, said method comprising administering a pseudohalide to said mammal such that said amount of NO is increased.

26. The method of claim 25, further comprising monitoring the level of said pseudohalide in said mammal.

27. The method of claim 25, wherein said pseudohalide is administered orally, nasally, or topically.

28. The method of claim 25, wherein said pseudohalide is administered by injection or inhalation.

29. The method of claim 25, wherein said administration comprises administering a pill or a dietary supplement containing said pseudohalide.

30. A bronchial inhaler device comprising an aerosolizable form of a pseudohalide.

31. The inhaler device of claim 30, wherein said pseudohalide is selected from the group consisting of thiocyanate, salts of thiocyanate, isothiocyanate, and salts of isothiocyanate.

32. A method of identifying a mammal that can benefit from treatment with a pseudohalide, said method comprising determining the level of said pseudohalide in a biological sample from said mammal and identifying said mammal as one that can benefit from treatment with said pseudohalide if said level of said pseudohalide is reduced relative to that of a control sample.

33. The method of claim 32, wherein said pseudohalide is selected from the group consisting of thiocyanate, salts of thiocyanate, isothiocyanate, and salts of isothiocyanate.

34. The method of claim 32, wherein said biological sample is selected from the group consisting of bronchoalveolar lavage, serum, plasma, urine, tissue biopsy, sputum, induced sputum, blood, pericardial fluid, pleural fluid, and cerebrospinal fluid.

35. A process for aiding in the diagnosis of a disease condition comprising
(a) manufacturing a diagnostic device comprising a thiocyanate-reactive reagent, wherein said thiocyanate-reactive reagent is capable of reacting with thiocyanate in a biological sample to form a detectable product; and
(b) selling said diagnostic device to an organization involved in managing or providing healthcare.

36. A process for aiding in the treatment of a disease condition comprising
(a) manufacturing a pharmaceutical composition comprising a pseudohalide having an oxidative reactivity with a peroxidase greater than that of bromide and a lower degree of toxicity than HOBr; and
(b) selling said pharmaceutical composition to an organization involved in managing or providing healthcare.

37. The process of claim 36, wherein said pseudohalide is selected from the group consisting of thiocyanate, salts of thiocyanate, isothiocyanate, and salts of isothiocyanate.

38. The use of a pseudohalide in the manufacture of a medicament for the treatment of eosinophil-mediated inflammation in mammal, wherein administration of said pseudohalide is effective for reducing eosinophil-mediated inflammation.

39. The use of claim 38, wherein said pseudohalide is selected from the group consisting of thiocyanate, salts of thiocyanate, isothiocyanate, and salts of isothiocyanate.
